(19) United States
(12) Patent Application Publication
(10) Pub. No.: US 2003/0190333 A1
(43) Pub. Date: Oct. 9, 2003

(54) Immunostimulant compositions comprising aminoalkyl glucosaminide phosphates and saponins

(75) Inventors: Sally Mossman, Seattle, WA (US); Lawrence Evans, Seattle, WA (US); Jory R. Baldridge, Victor, MT (US); Jay T. Evans, Corvallis, MT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation, Seattle, WA (US)

(21) Appl. No.: 10/068,171

(22) Filed: Feb. 4, 2002

(51) Int. Cl. A61K 39/02; A61K 31/739

(52) U.S. Cl. 424/234.1; 514/42; 514/54

(57) ABSTRACT

The invention provides pharmaceutical compositions, particularly vaccine compositions, employing an adjuvant system comprising at least one aminoalkyl glucosaminide phosphate compound and at least one saponin compound. Such compositions synergistically enhance the immune response in a mammal to a co-administered antigen. Also provided are methods of using the compositions in the treatment of various human diseases, including cancer, microbial infections and autoimmune disorders.

### FIELD OF THE INVENTION

The present invention relates generally to vaccine, adjuvant and immunostimulant formulations, to methods for their production and to their use in prophylactic and/or therapeutic vaccination. More particularly, the present invention relates to an adjuvant system comprising saponin compounds in combination with aminoalkyl glucosaminide phosphates.

### BACKGROUND OF THE INVENTION

Humoral immunity and cell-mediated immunity are the two major branches of the mammalian immune response. Humoral immunity involves the generation of antibodies to foreign antigens. Antibodies are produced by B-lymphocytes. Cell-mediated immunity involves the activation of T-lymphocytes which either act upon infected cells bearing foreign antigens or stimulate other cells to act upon infected cells. Both branches of the mammalian immune system are important in fighting disease. Humoral immunity is the major line of defense against bacterial pathogens. In the case of viral disease, the induction of cytotoxic T lymphocytes (CTLs) appears to be crucial for protective immunity. Thus, an effective vaccine preferably stimulates both branches of the immune system to protect against disease.

Vaccines present foreign antigens from disease causing agents to a host so that the host can mount a protective immune response. Often, vaccine antigens are killed or attenuated forms of the microbes which cause the disease. The presence of non-essential components and antigens in these killed or attenuated vaccines has encouraged considerable efforts to refine vaccine components including developing well-defined synthetic antigens using chemical and recombinant techniques. The refinement and simplification of microbial vaccines, however, has led to a concomitant loss in potency. Low-molecular weight synthetic antigens, though devoid of potentially harmful contaminants, are often not sufficiently immunogenic by themselves. These observations have led investigators to add immune system stimulators known as adjuvants to vaccine compositions to potentiate the activity of the vaccine components.

Immune adjuvants are compounds which, when administered to an individual or tested in vitro, increase the immune response to an antigen in a present invention to which the antigen is administered, or enhance certain activities of cells from the immune system. A number of compounds exhibiting varying degrees of adjuvant activity have been prepared and tested (see, for example, Shimizu et al. 1985, Bulusu et al. 1992, Ikeda et al. 1993, Shimizu et al. 1994, Shimizu et al. 1995, Miyajima et al. 1996). However, these and other prior adjuvant systems often display toxic properties, are unstable and/or have unacceptably low immunostimulatory effects.

The innate immune system coordinates the inflammatory response to pathogens by a system that discriminates between self and non-self via receptors that identify classes of molecules synthesized exclusively by microbes. These classes are sometimes referred to as pathogen associated molecular patterns (PAMPs) and include, for example, lipopolysaccharide (LPS), peptidoglycans, lipotechoic acids, and bacterial lipoproteins (BLPs).

LPS is an abundant outer cell-wall constituent from gram-negative bacteria that is recognized by the innate immune system. Although the chemical structure of LPS has been known for some time, the molecular basis of recognition of LPS by serum proteins and/or cells has only recently begun to be elucidated. In a series of recent reports, a family of receptors, referred to as Toll-like receptors (TLRs), have been linked to the potent innate immune response to LPS and other microbial components. All members of the TLR family are membrane proteins having a single transmembrane domain. The cytoplasmic domains are approximately 200 amino acids and share similarity with the cytoplasmic domain of the IL-1 receptor. The extracellular domains of the Toll family of proteins are relatively large (about 550-980 amino acids) and may contain multiple ligand-binding sites.

The importance of TLRs in the immune response to LPS has been specifically demonstrated for at least two Toll-like receptors, Tlr2 and Tlr4. For example, transfection studies with embryonic kidney cells revealed that human Tlr2 was sufficient to confer responsiveness to LPS (Yang et al., Nature 395:284-288 (1998); Kirschning et al. J Exp Med. 11:2091-97 (1998)). A strong response by LPS appeared to require both the LPS-binding protein (LBP) and CD14, which binds LPS with high affinity. Direct binding of LPS to Tlr2 was observed at a relatively low affinity, suggesting that accessory proteins may facilitate binding and/or activation of Tlr2 by LPS in vivo.

The importance of Tlr4 in the immune response to LPS was demonstrated in conjunction with positional cloning in lps mutant mouse strains. Two mutant alleles of the mouse lps gene have been identified, a semidominant allele that arose in the C3H/HeJ strain and a second, recessive allele that is present in the C57BL/10ScN and C57BL/10ScCr strains. Mice that are homozygous for mutant alleles of lps are sensitive to infection by Gram-negative bacteria and are resistant to LPS-induced septic shock. The lps locus from these strains was cloned and it was demonstrated that the mutations altered the mouse Tlr4 gene in both instances (Portorak et al., Science 282:2085-2088 (1998); Qureshi et al., J Exp Med 4:615-625 (1999)). It was concluded from these reports that Tlr4 was required for a response to LPS.

The biologically active endotoxic sub-structural moiety of LPS is lipid-A, a phosphorylated, multiply fatty-acid-acylated glucosamine disaccharide that serves to anchor the entire structure in the outer membrane of Gram-negative bacteria. We previously reported that the toxic effects of lipid A could be ameliorated by selective chemical modification of lipid A to produce monophosphoryl lipid A compounds (MPL® immunostimulant; Corixa Corporation; Seattle, Wash.). Methods of making and using MPL® immunostimulant and structurally like compounds in vaccine adjuvant and other applications have been described (see, for example, U.S. Pat. Nos. 4,436,727; 4,877,611; 4,866,034 and 4,912,094; 4,987,237; Johnson et al., J Med Chem 42:4640-4649 (1999); Ulrich and Myers, in Vaccine Design: The Subunit and Adjuvant Approach; Powell and Newman, Eds.; Plenum: New York, 495-524, 1995; the disclosures of which are incorporated herein by reference in their entireties). In particular, these and other references demonstrated that MPL® immunostimulant and related compounds had significant adjuvant activities when used in vaccine formulations with protein and carbohydrate antigens for enhancing humoral and/or cell-mediated immunity to the antigens.

A class of synthetic mono- and disaccharide mimetics of monophosphoryl lipid A, referred to as aminalkyl glucosaminide phosphates (AGPs), has been disclosed, for example in U.S. Pat. Nos. 6,113,918, and 6,303,347, U.S. patent application Ser. No. 09/074,720 filed May 7, 1998, and in PCT published application WO 98/50399, the disclosures of which are incorporated herein by reference in their entireties. Like monophosphoryl lipid A, these compounds have been demonstrated to retain significant adjuvant characteristics when formulated with antigens in vaccine compositions and, in addition, have similar or improved toxicity profiles when compared with monophosphoryl lipid A. A significant advantage offered by the AGPs is that they are readily producible on a commercial scale by synthetic means.

The discovery and development of effective adjuvant systems is essential for improving the efficacy and safety of existing and future vaccines. Thus, there is a continual need for new and improved adjuvant systems, particularly those that drive both effector arms of the immune system, to better facilitate the development of a next generation of synthetic vaccines. The present invention fulfills these and other needs.

### SUMMARY OF THE INVENTION

According to one aspect of the present invention, immunostimulant compositions are provided comprising at least one aminoalkyl glucosaminide phosphate (AGP) and at least one saponin compound.

AGP compounds employed in the compositions of the present invention may be monosaccharide or disaccharide compounds. Thus, the present invention provides immunostimulant compositions that comprise one or more AGP compounds having the formula:

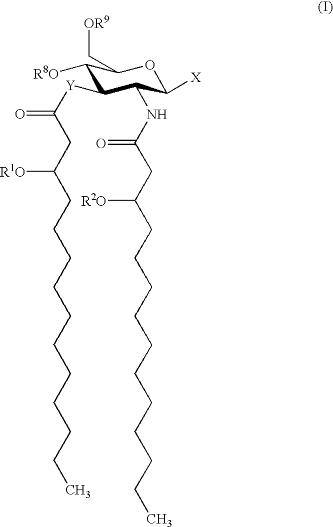

and pharmaceutically acceptable salts and derivatives thereof, wherein Y is —O— or —NH—; R¹ and R² are each independently selected from saturated and unsaturated (C₂-C₂₄) aliphatic acyl groups; R⁸ is —H or —PO₃R¹¹R¹², wherein R¹¹ and R¹² are each independently —H or (C₁-C₄) aliphatic groups; R⁹ is —H, —CH₃ or —PO₃R¹³R¹⁴, wherein R¹³ and R¹⁴ are each independently selected from —H and (C₁-C₄) aliphatic groups; and wherein at least one of R⁸ and R⁹ is a phosphorus-containing group, but R⁸ and R⁹ are not both phosphorus-containing groups; and X is a group selected from the formulae:

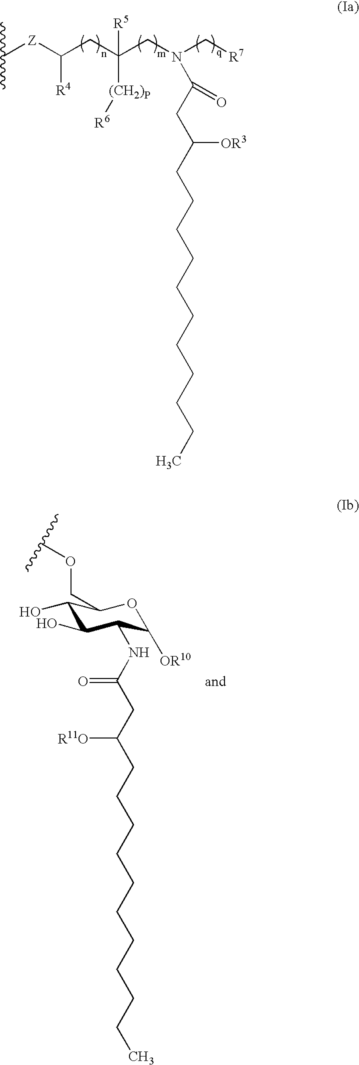

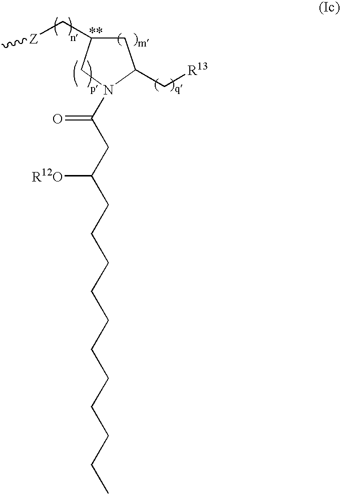

wherein the subscripts n, m, p, q, n′, m′, p′ and q′ are each independently an integer of from 0 to 6, provided that the sum of p′ and m′ is an integer from 0 to 6; R³, R¹¹, and R¹² are independently a saturated or unsaturated optionally substituted aliphatic (C₂-C₂₄)acyl group, provided that when X is formula (Ia), one of R¹, R² and R³ is optionally hydrogen; R⁴ and R⁵ are independently selected from H and methyl; R⁶ and R⁷ are independently selected from H, OH, (C₁-C₄)oxyaliphatic groups, —PO₃H₂, —OPO₃H₂, —SO₃H, —OSO₃H, —NR¹⁵R¹⁶, —SR¹⁵, —CN, —NO₂, —CHO, —CO₂R¹⁵, —CONR¹⁵R¹⁶, —PO₃R¹⁵R^{16, —OPO}₃R¹⁵R¹⁶, —SO₃R¹⁵ and —OSO₃R¹⁵, wherein R¹⁵ and R¹⁶ are each independently selected from H and (C₁-C₄) aliphatic groups; R¹⁰ is selected from H, CH₃, —PO₃H₂, ω-phosphonooxy(C₂-C₂₄)alkyl, and ω-carboxy(C₁-C₂₄)alkyl; R¹³ is independently selected from H, OH, (C₁-C₄)oxyaliphatic groups, —PO₃R¹⁷R¹⁸, —OPO₃R¹⁷R¹⁸, —SO₃R¹⁷, —OSO₃R¹⁷, —NR¹⁷R¹⁸, —SR¹⁷, —CN, —NO₂, —CHO, —CO₂R¹⁷, and —CONR¹⁷R¹⁸, wherein R¹⁷ and R¹⁸ are each independently selected from H and (C₁-C₄)aliphatic groups; and Z is —O— or —S—.

Within certain embodiments of the present invention, the AGP compounds employed in the immunostimulant compositions are those disclosed, for instance, in U.S. Pat. No. 6,113,918, which is hereby incorporated herein, and which generally conform to the following structure:

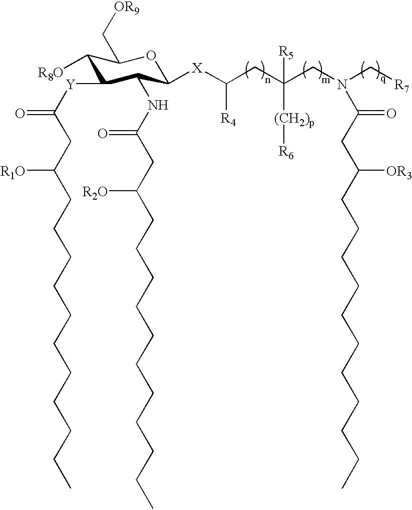

and pharmaceutically acceptable salts, derivatives and biologically active fragments thereof, wherein X represents an oxygen or sulfur atom, Y represents an oxygen atom or NH group, “n”,“m”, “p” and “q” are integers independently selected from 0 to 6, R₁, R₂, and R₃ represent fatty acyl residues, including saturated, unsaturated, and branched acyl groups, having 7 to 16 carbon atoms, R₄ and R₅ are independently selected from hydrogen and methyl, R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono.

Other compositions of the present invention employ AGP compounds that are disclosed in U.S. Pat. No. 6,303,347, which is hereby incorporated herein, and which generally conform to the following structure:

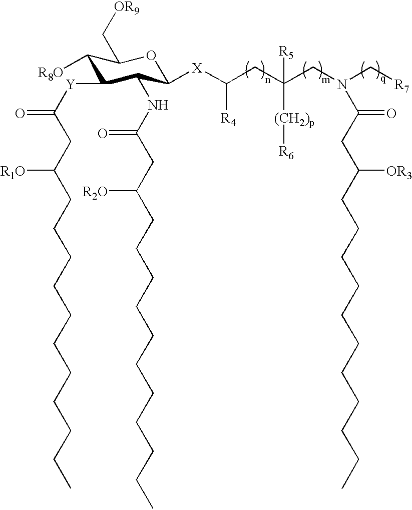

and pharmaceutically acceptable salts, derivatives and biologically active fragments thereof, wherein X represents an oxygen or sulfur atom in either the axial or equatorial position; Y represents an oxygen atom or NH group; “n”, “m”, “p” and “q” are integers independently selected from 0 to 6; R₁, R₂, and R₃ represent fatty acyl residues, including saturated, unsaturated, and branched acyl groups, having 1 to 20 carbon atoms and where one of R₁, R₂ or R₃ is optionally hydrogen; R₄ and R₅ are independently selected from hydrogen or methyl; R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono.

Still further exemplary embodiments of the present invention provide immunostimulant compositions that employ AGP compounds disclosed in PCT/US01/24284, filed Aug. 3, 2001, which application is incorporated herein by reference, and generally conform to the following structure:

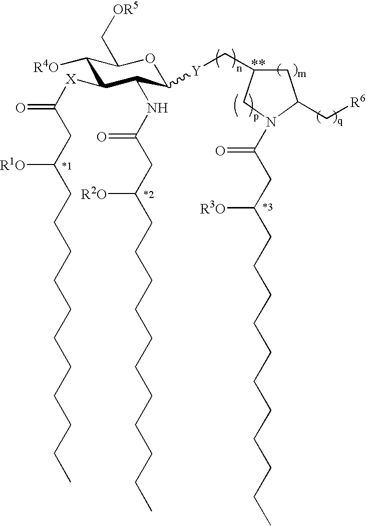

and pharmaceutically acceptable salts thereof, wherein X is a member selected from the group consisting of —O— and —NH—; Y is a member selected from the group consisting of —O— and —S—; R¹, R² and R³ are each members independently selected from the group consisting of (C₂-C₂₄)acyl; R⁴ is a member selected from the group consisting of —H and —PO₃R⁷R⁸, wherein R⁷ and R⁸ are each members independently selected from the group consisting of —H and (C₁-C₄)alkyl; R⁵ is a member selected from the group consisting of —H, —CH₃ and —PO₃R⁹R¹⁰, wherein R⁹ and R¹⁰ are each members independently selected from the group consisting of —H and (C₁-C₄)alkyl; R⁶ is selected from H, OH, (C₁-C₄)alkoxy, —PO₃R¹¹R¹², —OPO₃R¹¹R¹², —SO₃R¹¹, —OSO₃R¹¹, —NR¹¹R¹², —SR¹¹, —CN, —NO₂, —CHO, —CO₂R¹¹, and —CONR¹¹R¹², wherein R¹¹ and R¹² are each independently selected from H and (C₁-C₄)alkyl, with the provisos that one of R⁴ and R⁵ is a phosphorus-containing group and that when R⁴ is —PO₃R⁷R⁸, R⁵ is other than —PO₃R⁹R¹⁰; wherein “*1”, “*2”, “*3” and “**” represent chiral centers; wherein the subscripts n, m, p and q are each independently an integer from 0 to 6, with the proviso that the sum of p and m is from 0 to 6. Within certain embodiments, R¹, R² and R³ are each members independently selected from the group consisting of (C₉-C₁₆) acyl, or from the group consisting of (C₁₀-C₁₄) acyl, or from the group consisting of (C₁₀-C₁₂) acyl.

According to another embodiment of this invention, the AGP in the compositions of this invention is a monophosphoryl lipid A (MPL®, Corixa Corporation). MPL® is described in U.S. Pat. Nos. 4,436,727; 4,877,611; 4,866,034 and 4,912,094; 4,987,237; Johnson et al., J Med Chem 42:4640-4649 (1999); Ulrich and Myers, in Vaccine Design: The Subunit and Adjuvant Approach; Powell and Newman, Eds.; Plenum: New York, 495-524, 1995; the disclosures of which are incorporated herein by reference in their entireties.

The saponins that may be employed in the compositions of this invention include saponins (naturally or synthetically obtained), saponin conjugates, saponin derivatives, and saponin mimetics, all as described herein.

According to one aspect of the present invention, the saponin employed in the immunostimulant composition comprises a Quillaja saponin, e.g., QuilA and/or QS-21 (Aquila Biopharmaceuticals, Worcester, Mass. In one preferred embodiment of this aspect of the invention, the Quillaja saponin comprises QS-7, QS-17, QS-18 and/or QS-21.

According to another aspect of the present invention, the saponin employed in the immunostimulant composition comprises a triterpene saponin-lipophile conjugate comprising a nonacylated or desacylated triterpene saponin that includes a 3-glucuronic acid residue; and a lipophilic moiety; wherein said saponin and said lipophilic moiety are covalently attached to one another, either directly or through a linker group, and wherein said direct attachment or attachment to said linker occurs through a covalent bond between the carboxyl carbon of said 3-glucuronic acid residue, and a suitable functional group on the lipophilic residue or linker group. Some saponin-lipophile conjugates useful in this invention, including GPI-0100, a quilaja saponin-lipophile conjugate, are disclosed in U.S. Pat. Nos. 5,977,081 and 6,080,725, each of which is incorporated herein by reference in its entirety. Other saponin-lipophile conjugates are disclosed in U.S. Pat. No. 6,262,019, which is incorporated herein in its entirety.

The triterpene saponin can have a triterpene aglycone core structure with branched sugar chains attached to positions 3 and 28, and an aldehyde group linked or attached to position 4; and is either originally non-acylated, or requires removal of an acyl or acyloyl group that is bound to a saccharide at the 28-position of the triterpene aglycone. The triterpene saponin can have a quillaic acid or gypsogenin core structure.

The desacylsaponin or nonacylated saponin can be selected from the group consisting of Quillaja desacylsaponin, S. jenisseensis desacylsaponin, Gypsophila saponin, Saponaria saponin, Acanthophyllum saponin and lucyoside P saponin.

The lipophilic moiety can comprise one or more residues of a fatty acid, terpenoid, aliphatic amine, aliphatic alcohol, aliphatic mercaptan, mono- or poly-C₂-C₄ alkyleneoxy derivative of a fatty acid, mono- or poly-C₂-C₄ alkyleneoxy derivative of a fatty alcohol, glycosyl-fatty acid, glycolipid, phospholipid or a mono-, or di-acylglycerol.

In another aspect of the present invention, the saponin employed in the immunostimulant composition comprises a saponin/antigen covalent conjugate composition.

In another aspect of the present invention, the saponin employed in the immunostimulant composition comprises a saponin mimetic compound represented by the formula:

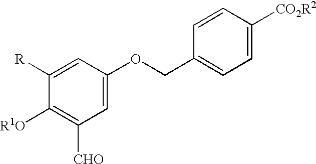

where the symbol R represents hydrogen or —C(O)H. The symbol R¹ represents a member selected from hydrogen, an optionally substituted C₁-C₂₀ aliphatic group, a saccharyl group, and a group represented by the formula —C(O)—[C(R³)(R⁴)]ₖ—COOH or —[C(R³)(R⁴)]ₖ—COOH, wherein each R³ and R⁴ independently is a member selected from hydrogen or an optionally substituted C₁₋₁₀ aliphatic group. The symbol “k” represents an integer from 1 to 5. The symbol R² represents a member selected from hydrogen, an optionally substituted C₁-C₂₀ aliphatic group, and a group represented by the formula —(CH₂)ᵣCH(OH)(CH₂)ₜOR⁵, wherein r and t are independently 1 or 2, and R⁵ is a C₂₋₂₀ acyl group, or a group represented by the formula

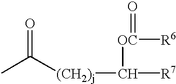

wherein j is an integer from 1 to 5, and R⁶ and R⁷ are independently selected from the group of hydrogen and optionally substituted C₁₋₂₀ aliphatic groups; or is a pharmacologically acceptable salt thereof. In another aspect of the present invention, the immunostimulant compositions described above further comprise at least one antigen.

Saponin mimetics of this type are disclosed in U.S. patent application Ser. No. 09/810,915 filed Mar. 16, 2001 of David A. Johnson, enitled “Novel Amphipathic Aldehydes and their Uses as Adjuvants and Immunoeffectors” and PCT application (publication no.) WO 01/70663, both of whch are hereby incorporated herein in their entireties.

According to another aspect of the invention, the immunostimulant compositions of the invention are formulated in a solid formulation, a stable emulsion formulation or an aqueous formulation.

According to another aspect of the present invention, there is provided a method of treating a mammal suffering from or susceptible to a pathogenic infection, cancer or an autoimmune disorder comprising administering to the mammal an effective amount of an immunostimulant composition of the present invention.

According to another aspect of the present invention, there is provided a method of enhancing the immune response in an animal that comprises administering to the animal an immunostimulant composition of the present invention.

According to another aspect of the present invention, there is provided a method of enhancing the immune response in an animal to an antigen that comprises administering to the animal an immunostimulant composition of the present invention in combination with an antigen.

### Definitions

The term “acyl” refers to those groups derived from an aliphatic organic acid by removal of the hydroxy portion of the acid. Accordingly, acyl is meant to include, for example, acetyl, propionyl, butyryl, decanoyl, pivaloyl, and the like. A “C₁-C₂₀ acyl group” thus is an acyl group having from 1 to 20 carbons.

The term “aliphatic,” means, unless otherwise stated, a non-aromatic straight or branched chain, or cyclic, hydrocarbon moiety, saturated or mono- or poly-unsaturated, including such a moiety that contains both cyclical and chain elements, having the designated number of carbon atoms (i.e. C₁-C₁₀ means having from one to ten carbons). Types of saturated hydrocarbon radicals include alkyl, alkylene, cycloalkyl or cycloalkyl-alkyl groups such as methyl, ethyl, n-propyl, isopropyl, n-butyl, t-butyl, isobutyl, sec-butyl, methylene, ethylene, n-butylene, cyclopropyl, and cyclopropylmethyl.

An unsaturated aliphatic group is one having one or more double and/or triple bonds. Examples of unsaturated aliphatic groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, cyclohexenyl, and cyclohexadienyl.

A “C₁-C₂₀ aliphatic group” is a substituted or unsubstituted aliphatic group having from 1 to 20 carbons. Similarly, a “C₁₁ aliphatic group” is a substituted or unsubstituted aliphatic group having 11 carbons.

The term “oxyaliphatic” refers to those aliphatic groups attached to the remainder of the molecule via an oxygen atom.

The terms “halo” or “halogen,” by themselves or as part of another substituent, mean, unless otherwise stated, a fluorine, chlorine, bromine, or iodine atom. In compounds having halogen substituents, the halogens may be the same or different.

Substituents for the aliphatic groups can be a variety of groups selected from: —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —NR′C(NR′R″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical and R′, R″and R′″each independently refer to hydrogen or (C₁-C₄)aliphatic groups. When a compound of the invention includes more than one R group, for example, each of the R groups is independently selected, as are each R′, R″ and R′″ groups when more than one of these groups is present. When R′ and R″ are attached to the same nitrogen atom, they can be combined with the nitrogen atom and optionally an additional heteroatom to form a 5-, 6-, or 7-membered ring. For example, —NR′R″ is meant to include 1-pyrrolidinyl and 4-morpholinyl. From the above discussion of substituents, one of skill in the art will understand that the term “aliphatic” is meant to include groups such as haloaliphatic (e.g., —CF₃, CClF₂, and —CH₂CF₃).

The term “saccharyl” refers to those groups derived from a sugar, a carbohydrate, a saccharide, a disaccharide, an oligosaccharide, or a polysaccharide molecule by removal of a hydrogen or a hydroxyl group. Accordingly, saccharyl groups (e.g., glucosyl, mannosyl, etc.) can be derived from molecules that include, but are not limited to, glucuronic acid, lactose, sucrose, maltose, allose, alltrose, glucose, mannose, idose, galactose, talose, ribose, arabinose, xylose, lyxose, threose, erythrose, β-D-N-Acetylgalactosamine, β-D-N-Acetylglucosamine, fucose, sialic acid, etc. A “C₆-C₂₀ saccharyl group” is a substituted (e.g. acylated saccharyl, alkylated saccharyl, arylated saccharyl, etc.) or unsubstituted saccharyl group having from 6 to 20 carbons. An example of a saccharyl group is a radical formed by the removal of the hydroxyl on the C1 position of glucuronic acid as represented by the formula:

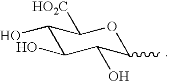

The wavy bond indicates where the glucuronide radical (i.e., a glucuronic acid group) would be attached to another substituent, e.g., an aglycon unit. Thus, saccharyl groups include sugar molecules where the hydroxyl on the C1 position has been removed.

The term “pharmaceutically acceptable salts” is meant to include salts of the compounds in question that are prepared with relatively nontoxic acids or bases, depending on the particular substituents found on the compounds described herein. When compounds of the present invention contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds of the present invention contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, compounds which are in a prodrug form of the saponins or aminoalkyl glucosaminide phosphates may be included in the compositions of this invention. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds usable in compositions of the present invention can exist in unsolvated forms as well as solvated forms, including hydrated forms. In general, the solvated forms are equivalent to unsolvated forms and are encompassed within the scope of the present invention. Certain compounds usable in compositions of the present invention may exist in multiple crystalline or amorphous forms. In general, all physical forms are equivalent for the uses contemplated by the present invention and are intended to be within the scope of the present invention.

Certain compounds usable in compositions of the present invention possess asymmetric carbon atoms (optical centers) or double bonds; the racemates, diastereomers, geometric isomers and individual isomers are encompassed within the scope of the present invention.

The chemical compounds in compositions of the present invention may exist in (+) and (−) forms as well as in racemic forms. Racemic forms can be resolved into the optical antipodes by known methods and techniques. One way of separating the racemic forms is exemplified by the separation of racemic amines by conversion of the racemates to diastereomeric salts of an optically active acid. The diastereomeric salts are resolved using one or more art recognized methods. The optically active amine is subsequently liberated by treating the resolved salt with a base. Another method for resolving racemates into the optical antipodes is based upon chromatography on an optical active matrix. Racemic compounds used in compositions of the present invention can thus be resolved into their optical antipodes, e.g., by fractional crystallization of d- or l-tartrates, -mandelates, or -camphorsulfonate) salts for example.

Such compounds may also be resolved by the formation of diastereomeric amides by reaction with an optically active carboxylic acid such as that derived from (+) or (−) phenylalanine, (+) or (−) phenylglycine, (+) or (−) camphanic acid or the like. Alternatively, they may be resolved by the formation of diastereomeric carbamates by reaction of the chemical compound with an optically active chloroformate or the like.

Additional methods for the resolving the optical isomers are known in the art. Such methods include those described by Collet and Wilen, ENANTIOMERS, RACEMATES, AND RESOLUTIONS, John Wiley and Sons, New York (1981).

Moreover, some of the compounds usable in compositions of the invention can exist in syn- and anti-forms (Z- and E-form), depending on the arrangement of the substituents around a double bond. A chemical compound in a composition of the present invention may thus be the syn- or the anti-form (Z- and E-form), or it may be a mixture thereof.

The compounds usable in these compositions may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of such compounds, whether radioactive or not, are intended to be encompassed within the scope of the present invention.

An “effective immunopotentiatory amount” is an amount of a compound or composition that is effective to potentiate an immune response to one or more antigens. The immune response can be measured, without limitation, by measuring antibody titers against an antigen (e.g., HBsAg, etc.), assessing the ability of a vaccine containing a compound of the present invention to immunize a host in response to a disease or antigen challenge, etc. Preferably, administering an “effective immunopotentiatory amount” of a compound or composition to a subject increases one or more antibody titers (e.g., IgG1a, IgG1b, IgG2a, IgG2b, etc.) by 10% or more over a nonimmune control, even more preferably by 20% or more over a nonimmune control, and still more preferably by 30% or more over a nonimmune control, and most preferably by 100% or more over a nonimmune control.

### DETAILED DESCRIPTION OF THE INVENTION

### I. Introduction

The present invention involves compositions that comprise at least one aminoalkyl glucosaminide phosphate (AGP) and at least one saponin compound (both as defined herein). These compositions are useful as immunostimulants when administered to subjects. In certain embodiments, these immunostimulants are administered with vaccines.

### II. Aminoalkyl Glucosaminide Phosphates (AGPs)

Aminoalkyl glucosaminide phosphate (AGP) compounds generally comprise a 2-deoxy-2-amino-α-D-glucopyranose (glucosaminide) in glycosidic linkage with an aminoalkyl (aglycon) group. Suitable AGP compounds, and methods for their synthesis and use, are described generally in U.S. Pat. Nos. 6,113,918 and 6,303,347, WO 98/50399, U.S. patent application Ser. No. 09/074,720 filed May 7, 1998, International patent application PCT/US01/24284, and Johnson et al. (1999) Bioorg. Med. Chem. Lett. 9: 2273-2278, the disclosures of which are incorporated herein by reference in their entirety.

AGP compounds employed in the compositions of the present invention may be monosaccharide or disaccharide compounds. Thus, the present invention provides immunostimulant compositions that comprise one or more AGP compounds having the formula:

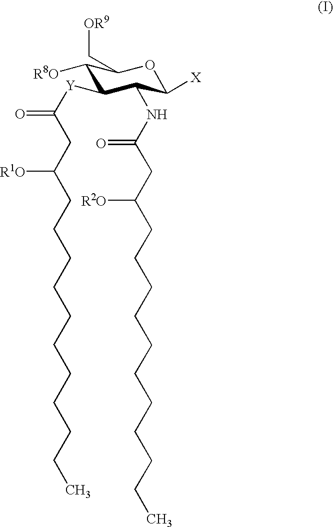

and pharmaceutically acceptable salts and derivatives thereof, wherein Y is —O— or —NH—; R¹ and R² are each independently selected from saturated and unsaturated (C₂-C₂₄) aliphatic acyl groups; R⁸ is —H or —PO₃R¹¹R¹², wherein R¹¹ and R¹² are each independently —H or (C₁-C₄) aliphatic groups; R⁹ is —H, —CH₃ or —PO₃R¹³R¹⁴, wherein R¹³ and R¹⁴ are each independently selected from —H and (C₁-C₄) aliphatic groups; and wherein at least one of R⁸ and R⁹ is a phosphorus-containing group, but R⁸ and R⁹ are not both phosphorus-containing groups; and X is a group selected from the formulae:

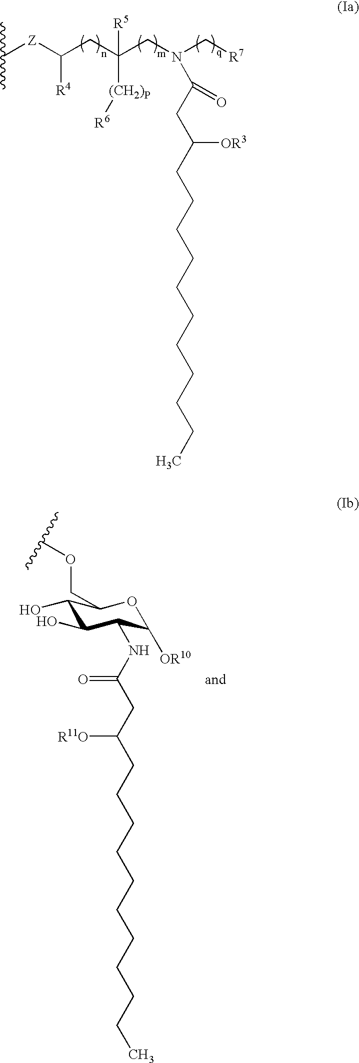

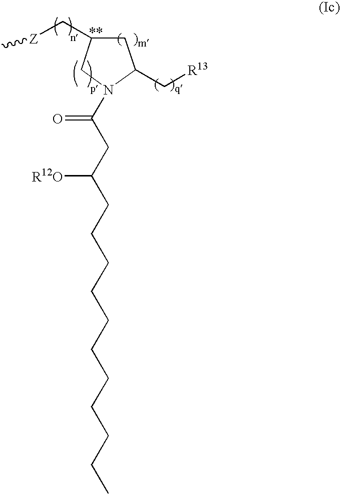

wherein the subscripts n, m, p, q, n′, m′, p′ and q′ are each independently an integer of from 0 to 6, provided that the sum of p′ and m′ is an integer from 0 to 6; R³, R¹¹, and R¹² are independently a saturated or unsaturated optionally substituted aliphatic (C₂-C₂₄)acyl group, provided that when X is formula (Ia), one of R¹, R² and R³ is optionally hydrogen; R⁴ and R⁵ are independently selected from H and methyl; R⁶ and R⁷ are independently selected from H, OH, (C₁-C₄) oxyaliphatic groups, —PO₃H₂, —OPO₃H₂, —SO₃H, —OSO₃H, —NR¹⁵R¹⁶, —SR¹⁵, —CN, —NO₂, —CHO, —CO₂R¹⁵, —CONR¹⁵R¹⁶, —PO₃R¹⁵R¹⁶, —OPO₃R¹⁵R¹⁶, —SO₃R¹⁵ and —OSO₃R¹⁵, wherein R¹⁵ and R¹⁶ are each independently selected from H and (C₁-C₄) aliphatic groups; R¹⁰ is selected from H, CH₃, —PO₃H₂, ω-phosphonooxy(C₂-C₂₄)alkyl, and ω-carboxy(C₁-C₂₄)alkyl; R¹³ is independently selected from H, OH, (C₁-C₄) oxyaliphatic groups, —PO₃R¹⁷R¹⁸, —OPO₃R¹⁷R¹⁸, —SO₃R¹⁷, —OSO₃R¹⁷, —NR¹⁷R¹⁸, —SR¹⁷, —CN, —NO₂, —CHO, —CO₂R¹⁷, and —CONR¹⁷R¹⁸, wherein R¹⁷ and R¹⁸ are each independently selected from H and (C₁-C₄) aliphatic groups; and Z is —O— or —S—.

One type of AGP compound of the present invention can be described generally by te following structure:

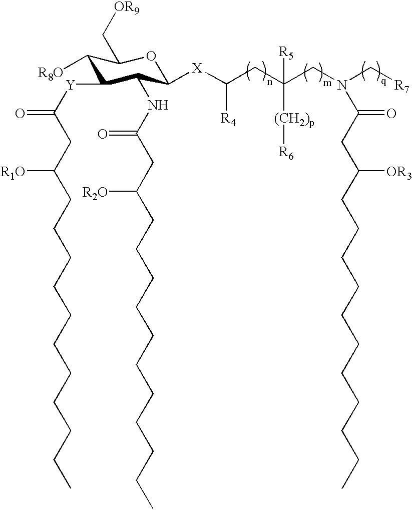

and pharmaceutically acceptable salts, derivatives and biologically active fragments thereof, wherein X represents an oxygen or sulfur atom, Y represents an oxygen atom or NH group, “n”, “m”, “p” and “q” are integers independently selected from 0 to 6, R₁, R₂, and R₃ represent fatty acyl residues, including saturated, unsaturated, and branched acyl groups, having 7 to 16 carbon atoms, R₄ and R₅ are independently selected from hydrogen and methyl, R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono. The configuration of the 3′ stereogenic centers to which the normal fatty acyl residues are attached is R or S, but preferably R. The stereochemistry of the carbon atoms to which R₄ or R₅ are attached can be R or S. All stereoisomers, both enantiomers and diastereomers, and mixtures thereof, are considered to fall within the scope of the present invention. See, U.S. Pat. No. 6,113,918.

Alternatively, AGP compounds employed in the immunostimulant compositions may generally conform to the following structure:

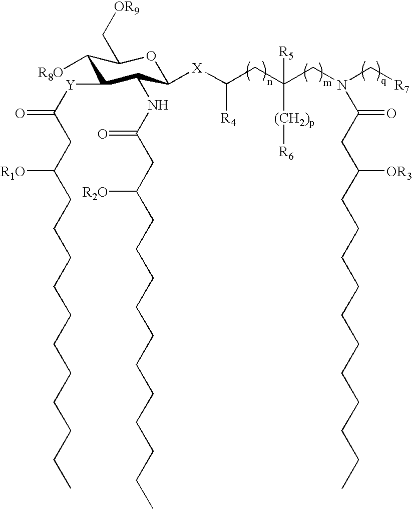

and pharmaceutically acceptable salts, derivatives and biologically active fragments thereof, wherein X represents an oxygen or sulfur atom in either the axial or equatorial position; Y represents an oxygen atom or NH group; “n”, “m”, “p” and “q” are integers independently selected from 0 to 6; R₁, R₂, and R₃ represent fatty acyl residues, including saturated, unsaturated, and branched acyl groups, having 1 to 20 carbon atoms and where one of R₁, R₂ or R₃ is optionally hydrogen; R₄ and R₅ are independently selected from hydrogen or methyl; R₆ and R₇ are independently selected from hydrogen, hydroxy, alkoxy, phosphono, phosphonooxy, sulfo, sulfooxy, amino, mercapto, cyano, nitro, formyl or carboxy and esters and amides thereof; R₈ and R₉ are independently selected from phosphono or hydrogen, wherein at least one of R₈ and R₉ is phosphono. See, U.S. Pat. No. 6,303,347.

Still further AGP compounds generally conform to the following structure:

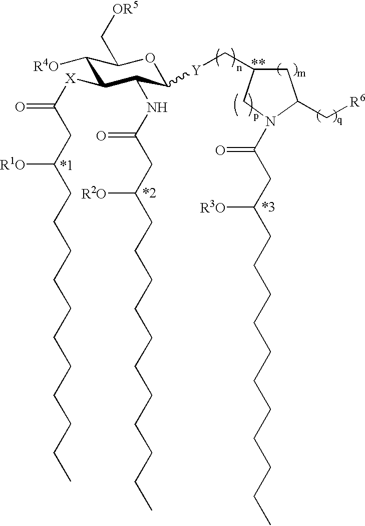

and pharmaceutically acceptable salts thereof, wherein X is a member selected from the group consisting of —O— and —NH—; Y is a member selected from the group consisting of —O— and —S—; R¹, R² and R³ are each members independently selected from the group consisting of (C₂-C₂₄)acyl; R⁴ is a member selected from the group consisting of —H and —PO₃R⁷R⁸, wherein R⁷ and R⁸ are each members independently selected from the group consisting of —H and (C₁-C₄)alkyl; R⁵ is a member selected from the group consisting of —H, —CH₃ and —PO₃R⁹R¹⁰, wherein R⁹ and R¹⁰ are each members independently selected from the group consisting of —H and (C₁-C₄)alkyl; R⁶ is selected from H, OH, (C₁-C₄)alkoxy, —PO₃R¹¹R¹², —OPO₃R¹¹R¹², —SO₃R¹¹, —OSO₃R¹¹, —NR¹¹R¹², —SR¹¹, —CN, —NO₂, —CHO, —CO₂R¹¹, and —CONR¹¹R¹², wherein R¹¹ and R¹² are each independently selected from H and (C₁-C₄)alkyl, with the provisos that one of R⁴ and R⁵ is a phosphorus-containing group and that when R⁴is —PO₃R⁷R⁸, R⁵is other than —PO₃R⁹R¹⁰; wherein “*1”, “*2”, “*3” and “**” represent chiral centers; wherein the subscripts n, m, p and q are each independently an integer from 0 to 6, with the proviso that the sum of p and m is from 0 to 6. See, PCT/US01/24284, filed Aug. 3, 2001. Within certain embodiments, R¹, R² and R³ are each members independently selected from the group consisting of (C₉-C₁₆) acyl, or from the group consisting of (C₁₀-C₁₄) acyl, or from the group consisting of (C₁₀-C₁₂) acyl. The heteroatoms X and Y of the AGP compounds can be oxygen or sulfur or —NH, as indicated. In a preferred embodiment, X is oxygen and typically in the equatorial position. Although the stability of the molecules could be affected by a substitution at X, the immunomodulating activity of molecules with these substitutions is not expected to change.

The number of carbon atoms between heteroatom X and the aglycon nitrogen atom is determined by variables “n” and “m”. Variables “n” and “m” can be integers from 0 to 6. In a preferred embodiment, the total number of carbon atoms between heteroatom X and the aglycon nitrogen atom is from about 2 to about 6 and most preferably from about 2 to about 4.

The AGPs are phosphorylated, such as at position 4 or 6 (formula Ia, R₈ or R₉) on the glucosaminide ring. For example, in one illustrative AGP of formula (Ia), R₈ is phosphono and R₉ is hydrogen. In one embodiment, the AGPs are hexaacylated, that is they contain a total of six fatty acid residues. The aminoalkyl glucosaminide moiety is acylated at the 2-amino and 3-hydroxyl groups of the glucosaminide unit and at the amino group of the aglycon unit with 3-hydroxyalkanoyl residues. In Formula (Ia), these three positions are acylated with 3-hydroxytetradecanoyl moieties. The 3-hydroxytetradecanoyl residues are, in turn, substituted with normal fatty acids (R₁-R₃), providing three 3-n-alkanoyloxytetradecanoyl residues or six fatty acid groups in total.

In another embodiment, the AGP compounds are pentaacylated, that is they contain a total of five fatty acid residues. More specifically, the 3-hydroxytetradecanoyl residues of Formula (Ia) are substituted with normal fatty acids at two of the three R₁, R₂ and R₃ positions, with the third R₁, R₂ or R₃ position being hydrogen. In other words, at least one of —OR₁, —OR₂ or —OR₃ is hydroxyl.

The chain length of normal fatty acids R₁-R₃ in the AGPs can be from 2 to about 24, and typically from about 7 to about 16 carbons. Preferably, R₁-R₃ are from about 9 to about 14 carbons. The chain lengths of these normal fatty acids can be the same or different. Although, only normal fatty acids are described, it is expected that unsaturated fatty acids (i.e. fatty acid moieties having double or triple bonds) substituted at R₁-R₃ on the compounds of the present invention would produce biologically active molecules. Further, slight modifications in the chain length of the 3-hydroxyalkanoyl residues are not expected to dramatically effect biological activity.

Preferred embodiments of the invention include compositions containing AGP compounds as defined above and methods of use of such compositions, having one or more of the following:

R¹, R², R³, R¹¹ and R¹² preferably are (C₇-C₁₆) aliphatic acyl groups, more preferably (C₈-C₁₄) aliphatic acyl groups, even more preferably (C₉-C₁₄) aliphatic acyl groups, yet even more preferably (C₁₀-C₁₄) aliphatic acyl groups, and most preferably are (C₁₀-C₁₄) saturated aliphatic acyl groups;

X is formula (Ia) and R¹, R², and R³ are all acyl groups (i.e., the compounds are hexa-acylated);

X is formula (Ia) and one of R¹, R² and R³ is hydrogen (i.e. the compounds are penta-acylated);

Z is oxygen;

when R⁸ or R⁹ is a phosphorus-containing group, such group preferably is an unsubstituted phosphoro group (R¹¹ and R¹², or R¹³ and R¹⁴, respectively, are both hydrogen); more preferably R⁸ is a phosphorus-containing group and R⁹ is hydrogen;

the total of n+m is an integer from 0 to 4, most preferably 0, 1 or 2;

p and q are independently 0, 1 or 2;

n′, m′, p′ and q′ are preferably independently an integer from 0 to 3; more preferably 0, 1, or 2; and most preferably n′ is 1, m′ is 2 and p′ and q′ are both 0 [i.e., the compounds of this type, where Y is formula (Ic), have a 2-pyrrolidinylmethyl configuration]

Another type of AGP usable in compositions of this invention is monophosphoryl lipid A (MPL®). MPL® is described in U.S. Pat. Nos. 4,436,727; 4,877,611; 4,866,034 and 4,912,094; 4,987,237; Johnson et al., J Med Chem 42:4640-4649 (1999); Ulrich and Myers, in Vaccine Design: The Subunit and Adjuvant Approach; Powell and Newman, Eds.; Plenum: New York, 495-524, 1995; the disclosures of which are incorporated herein by reference in their entireties. MPL often is in the form of a mixture of compounds that contains a mixture of disaccharides, some of which are of the formula (Ib), and some of which have a structure similar to formula (Ib) but have lesser degrees of acylation.

The following are illustrative subtypes of AGP compounds of formula (Ia).

In one illustrative class of such AGPs, R₆ is carboxy, X is O; Y is O; n, m, p and q are 0; R₁, R₂ and R₃ are normal fatty acyl residues having 10 carbon atoms; R₄, R₅ and R₇ are H; R⁸ is phosphono; R₉ is H; R₁, R₂ and R₃ are each attached to a stereogenic center having an R configuration; and R₅ is attached to a stereogenic center having an S configuration.

In another illustrative class of such AGPs, R₆ is carboxy, X is O; Y is O; n, m, p and q are 0; R₁, R₂ and R₃ are normal fatty acyl residues having 12 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is carboxy, X is O; Y is O; n, m, p and q are 0; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an R configuration.

In another illustrative class of such AGPs, R₆ is carboxy, X is O; Y is O; n, m, p and q are 0; R₁, R₂ and R₃ are normal fatty acyl residues having 8 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is H, X is O; Y is O; n is 2; m, p and q are 0; R₁, R₂ and R₃ are normal fatty acyl residues having 14 carbon atoms; R₄, R₅ and R₇ are H; R₈ is phosphono; R₉ is H; and R₁, R₂ and R₃ are each attached to a stereogenic center having an R configuration.

In another illustrative class of such AGPs, R₆ is H, X is O; Y is O; n is 1, m and p are 0; q is 1; R₁, R₂ and R₃ are normal fatty acyl residues having 10 carbon atoms; R₄ and R₅ are H; R₇ is carboxy; R₈ is phosphono; R₉ is H; and R₁, R₂ and R₃ are each attached to a stereogenic center having an R configuration.

In another illustrative class of such AGPs, R₆ is H, X is O; Y is O; m, n, p and q are 0; R₁, R₂ and R₃ are normal fatty acyl residues having 14 carbon atoms; R₄, R₅ and R₇ are H; R₈ is phosphono; R₉ is H; and R₁, R₂ and R₃ are each attached to a stereogenic center having an R configuration.

In another illustrative class of such AGPs, R₆ is H, X is O; Y is O; m, n, p and q are 0; R₁, R₂ and R₃ are normal fatty acyl residues having 10 carbon atoms; R₄, R₅ and R₇ are H; R₈ is phosphono; R₉ is H; and R₁, R₂ and R₃ are each attached to a stereogenic center having an R configuration.

In another illustrative class of such AGPs, R₆ is H, X is O; Y is O; m, p and q are 0; n is 1; R₁, R₂ and R₃ are normal fatty acyl residues having 14 carbons; R₄, R₅ and R₇ are H; R₈ is phosphono; R₉ is H; and R₁, R₂ and R₃ are each attached to a stereogenic center having an R configuration.

In another illustrative class of such AGPs, R₆ is hydroxy, X is O; Y is O; m, n and q are 0; p is 1; R₁, R₂ and R₃ are normal fatty acyl residues having 12 carbon atoms; R₄ and R₅ are H; R₇ is H; R₈ is phosphono; and R₉ is H; R₁, R₂ and R₃ are each attached to a stereogenic center having an R configuration; and R₅ is attached to a stereogenic center having an S configuration.

In another illustrative class of such AGPs, R₆ is hydroxy, X is O; Y is O; m and q are 0; n and p are 1; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, X is O; Y is O; m, n and q are 0; p is 2; R₁, R₂ and R₃ are normal fatty acyl residues having 10 carbon atoms; R₄, R₅ and R₇ are H; R⁸ is phosphono; R₉ is H; R₁, R₂ and R₃ are each attached to a stereogenic center having an R configuration; and R₅ is attached to a stereogenic center having an S configuration.

In another illustrative class of such AGPs, R is hydroxy, X is O; Y is O; m, n and q are 0; p is 1; R₁, R₂ and R₃ are normal fatty acyl residues having 14 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an R configuration.

In another illustrative class of such AGPs, R₆ is hydroxy, X is O; Y is O; m, n and q are 0; p is 1; R₁, R₂ and R₃ are normal fatty acyl residues having 14 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, X is O; Y is O; m, n and q are 0; p is 1; R₁, R₂ and R₃ are normal fatty acyl residues having 11 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, R₆ is hydroxy, X is O; Y is O; m, n and q are 0; p is 1; R₁, R₂ and R₃ are normal fatty acyl residues having 10 carbon atoms; R₄, R₅ and R₇ are H; R₈ is phosphono; R₉ is H; R₁, R₂ and R₃ are each attached to a stereogenic center having an R configuration; and R₅ is attached to a stereogenic center having an S configuration.

In another illustrative class of such AGPs, X is O; Y is O; m, n, p and q are 0; R₁, R₂ and R₃ are normal fatty acyl residues having 10 carbon atoms; R₄ and R₅ are H; R₆ is amino carbonyl; R₇ is H; R₈ is phosphono; and R₉ is H; R₁, R₂ and R₃ are each attached to a stereogenic center having an R configuration; and R₅ is attached to a stereogenic center having an S configuration.

In one particularly preferred embodiment of the invention, the AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-β-D-glucopyranoside triethylammonium salt. This corresponds to a compound having the structure set forth in Formula (Ia) in which R₁=R₂=R₃=n-C₁₃H₂₇CO, X=Y=O, n=m−p=q=0, R₄=R₅=R₆=R₇=R₉=H, and R₈=PO₃H₂, and is referred to in the “Examples” section compound B19.

In additional embodiments of the invention, preferred AGP compounds of Formula (Ia) include the following:

| | | | | | | |
|---|---|---|---|---|---|---|
| Ref. No. | R₁-R₃ | n | p | R₆ | q | R₇ |

| | | | | | | |
|---|---|---|---|---|---|---|
| B2** | n-C₁₃H₂₇CO | 0 | 1 | OH | 0 | H |
| B3 | n-C₁₁H₂₃CO | 0 | 1 | OH | 0 | H |
| B9 | n-C₉H₁₉CO | 1 | 1 | OH | 0 | H |
| B14** | n-C₉H₁₉CO | 0 | 0 | CO₂H | 0 | H |
| B15* | n-C₉H₁₉CO | 0 | 0 | CO₂H | 0 | H |
| B21 | n-C₁₃H₂₇CO | 1 | 0 | H | 0 | H |
| B22 | n-C₁₃H₂₇CO | 2 | 0 | H | 0 | H |
| B25 | n-C₉H₁₉CO | 0 | 0 | CONH₂ | 0 | H |

For all compounds shown: X = Y = O; R₄ = R₅ = H; m = 0; R₈ = phosphono; R₉ = H.
*the stereochemistry of the carbon atom to which R₅ is attached is S.
**the stereochemistry of the carbon atom to which R₅ is attached is R.

In yet another embodiment, an AGP of Formula (Ia) is:

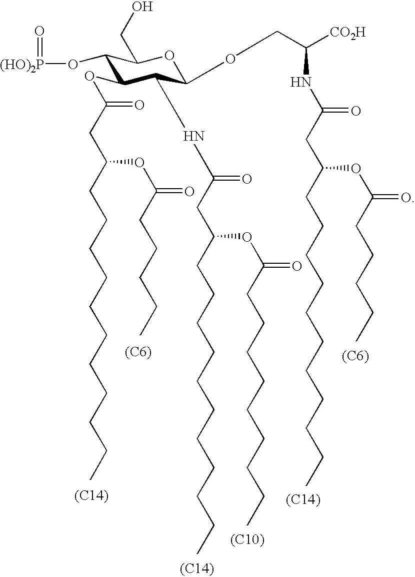

### III. Saponins

“Saponin,” as the term is used herein, encompasses natural and synthetic glycosidic triterpenoid compounds and pharmaceutically acceptable salts, derivatives, mimetics (e.g., isotucaresol and its deriviatives) and/or biologically active fragments thereof, which possess immune adjuvant activity.

In one illustrative embodiment, saponins employed in the vaccine compositions of the present invention can be purified from Quillaja saponaria Molina bark, as described in U.S. Pat. No. 5,057,540, the disclosure of which is incorporated herein by reference in its entirety.

The adjuvant properties of saponins were first recognized in France in the 1930's. (see, Bomford et al., Vaccine 1992, 10: 572-577). Two decades later the saponin from the bark of the Quillaja saponaria Molina tree found wide application in veterinary medicine, but the variability and toxicity of these crude preparations precluded their use in human vaccines. (see, Kensil et al., In Vaccine Design: The Subunit and Adjuvant Approach; Powell, M. F., Newman, J. J., Eds.; Plenum Press: New York, 1995 pp. 525-541).

In the 1970's a partially purified saponin fraction known as Quil A was shown to give reduced local reactions and increased potency (see, Kensil et al., 1995). Further fractionation of Quil A, which consisted of at least 24 compounds by HPLC, demonstrated that the four most prevalent saponins, QS-7, QS-17, QS-18, and QS-21, were potent adjuvants (see, Kensil, C. R. Crit Rev. Ther. Drug Carrier Syst. 1996, 13, 1-55; Kensil et al., 1995). QS-21 and QS-7 were the least toxic of these. Partly because of its reduced toxicity, highly purified state (though still a mixture of no less than four compounds), (see, Soltysik, S.; Bedore, D. A.; Kensil, C. R. Ann. N.Y. Acad. Sci. 1993, 690: 392-395) and more complete structural characterization, QS-21 (3) was the first saponin selected to enter human clinical trials. (see, Kensil, 1996; Kensil et al., 1995).

QS-21 and other Quillaja saponins increase specific immune responses to both soluble T dependent and T-independent antigens, promoting an Ig subclass switch in B-cells from predominantly IgG1 or IgM to the IgG2a and IgG2b subclasses (Kensil et al., 1995). The IgG2a and IgG2b isotypes are thought to be involved in antibody dependent cellular cytotoxicity and complement fixation (Snapper and Finkelman, In Fundamental Immunology, 4th ed.; Paul, W. E., Ed.: Lippincott-Raven: Philadelphia, Pa., 1999, pp. 831-861). These antibody isotypes also correlate with a Th-1 type response and the induction of IL-2 and IFN-γ-cytokines which play a role in CTL differentiation and maturation (Constant and Bottomly, Annu. Rev. Immunology 1997, 15: 297-322). As a result, QS-21 and other Quillaja saponins are potent inducers of class I MHC-restricted CD8+ CTLs to subunit antigens (Kensil, 1996; Kensil et al., 1995).

According to an aspect of the present invention, a saponin employed in the immunostimulant composition comprises a Quillaja saponin. In one preferred embodiment of this aspect of the invention, the Quillaja saponin comprises QS-7, QS-17, QS-18 and/or QS-21.

According to another aspect of the present invention, a saponin employed in the immunostimulant composition comprises a triterpene saponin-lipophile conjugate comprising a nonacylated or desacylated triterpene saponin that includes a 3-glucuronic acid residue; and a lipophilic moiety; wherein said saponin and said lipophilic moiety are covalently attached to one another, either directly or through a linker group, and wherein said direct attachment or attachment to said linker occurs through a covalent bond between the carboxyl carbon of said 3-glucuronic acid residue, and a suitable functional group on the lipophilic residue or linker group.

The triterpene saponin can have a triterpene aglycone core structure with branched sugar chains attached to positions 3 and 28, and an aldehyde group linked or attached to position 4; and is either originally non-acylated, or require removal of an acyl or acyloyl group that is bound to a saccharide at the 28-position of the triterpene aglycone. The triterpene saponin can have a quillaic acid or gypsogenin core structure. Some saponin-lipophile conjugates useful in this invention, including GPI-0100, a quilaja saponin-lipophile conjugate, are disclosed in U.S. Pat. Nos. 5,977,081 and 6,080,725, each of which is incorporated herein by reference in its entirety. The desacylsaponin or nonacylated saponin can be selected from the group consisting of Quillaja desacylsaponin, S. jenisseensis desacylsaponin, Gypsophila saponin, Saponaria saponin, Acanthophyllum saponin and lucyoside P saponin.

The lipophilic moiety can comprise one or more residues of a fatty acid, terpenoid, aliphatic amine, aliphatic alcohol, aliphatic mercapto mono- or poly-C₂-C₄ alkyleneoxy derivative of a fatty acid, mono- or poly-C₂-C₄ alkyleneoxy derivative of a fatty alcohol, glycosyl-fatty acid, glycolipid, phospholipid or a mono-, or di-acylglycerol.

In another aspect of the present invention, the saponin employed in the immunostimulant composition comprises a saponin/antigen covalent conjugate composition.

QS-21 and other Quillaja saponins can be purified from Quillaja sponaria using standard biochemical methodologies. Briefly, aqueous extracts of Quillaja saponaria Molina bark are dialyzed against water. The dialyzed extract is lyophilized to dryness, extracted with methanol, and the methanol-soluble extract is further fractionated on silica gel chromatography and by reverse phase high pressure liquid chromatography (RP-HPLC). The individual saponins are then be separated by reverse phase HPLC. At least 22 peaks (denominated QA-1 to QA-22, also referred to herein as QS-1 to QS-21) are separable using this approach, with each peak corresponding to a carbohydrate peak and exhibiting a single band on reverse phase thin layer chromatography. The individual components can be specifically identified by their retention times on a C4 HPLC column, for example.

Preferably, the Quillaja saponins employed according to this embodiment of the invention correspond to peaks QS-7, QS-17, QS-18, and/or QS-21, as described in U.S. Pat. No. 5,057,540. In one specific embodiment of the invention, QS-21 saponin is used in accordance with this disclosure.

The substantially pure QS-7 saponin is characterized as having immune adjuvant activity and containing about 35% carbohydrate (as assayed by anthrone) per dry weight. QS-7 has a UV absorption maxima of 205-210 nm, a retention time of approximately 9-10 minutes on RP-HPLC on a Vydac C₄ column having 5 μm particle size, 330 angstrom pore, 4.6 mm ID×25 cm L in a solvent of 40 mM acetic acid in methanol/water (58/42; v/v) at a flow rate of 1 ml/min, eluting with 52-53% methanol from a Vydac C₄ column having 5 μm particle size, 330 angstrom pore, 10 mM ID×25 cm L in a solvent of 40 mM acetic acid with gradient elution from 50 to 80% methanol, having a critical micellar concentration of approximately 0.06% in water and 0.07% in phosphate buffered saline, causing no detectable hemolysis of sheep red blood cells at concentrations of 200 μg/ml or less, and containing the monosaccharide residues terminal rhamnose, terminal xylose, terminal glucose, terminal galactose, 3-xylose, 3,4-rhamnose, 2,3-fucose, and 2,3-glucuronic acid, and apiose.

The substantially pure QS-17 saponin is characterized as having adjuvant activity and containing about 29% carbohydrate (as assayed by anthrone) per dry weight. QS-17 has a UV absorption maxima of 205-210 nm, a retention time of approximately 35 minutes on RP-HPLC on a Vydac C₄ column having 5 μm particle size, 330 angstrom pore, 4.6 mm ID×25 cm L in a solvent of 40 mM acetic acid in methanol-water (58/42; v/v) at a flow rate of 1 ml/min, eluting with 63-64% methanol from a Vydac C₄ column having 5 μm particle size, 330 angstrom pore, 10 mm ID×25 cm L in a solvent of 40 mM acetic acid with gradient elution from 50 to 80% methanol, having a critical micellar concentration of 0.06% (w/v) in water and 0.03% (w/v) in phosphate buffered saline, causing hemolysis of sheep red blood cells at 25 μg/ml or greater, and containing the monosaccharide residues terminal rhamnose, terminal xylose, 2-fucose, 3-xylose, 3,4-rhamnose, 2,3-glucuronic acid, terminal glucose, 2-arabinose, terminal galactose and apiose.

The substantially pure QS-18 saponin is characterized as having immune adjuvant activity and containing about 25-26% carbohydrate (as assayed by anthrone) per dry weight. QS-18 has a UV absorption maxima of 205-210 nm, a retention time of approximately 38 minutes on RP-HPLC on a Vydac C₄ column having 5 μm particle size, 330 angstrom pore, 4.6 mm ID×25 cm L in a solvent of 40 mM acetic acid in methanol/water (58/42; v/v) at a flow rate of 1 ml/min, eluting with 64-65% methanol from a Vydac C₄ column having 5 μm particle size, 330 angstrom pore, 10 mm ID×25 cm L in a solvent of 40 mM acetic acid with gradient elution from 50 to 80% methanol, having a critical micellar concentration of 0.04% (w/v) in water and 0.02% (w/v) in phosphate buffered saline, causing hemolysis of sheep red blood cells at concentrations of 25 μg/ml or greater, and containing the monosaccharides terminal rhamnose, terminal arabinose, terminal apiose, terminal xylose, terminal glucose, terminal galactose, 2-fucose, 3-xylose, 3,4-rhamnose, and 2,3-glucuronic acid.

The substantially pure QS-21 saponin is characterized as having immune adjuvant activity and containing about 22% carbohydrate (as assayed by anthrone) per dry weight. The QS-21 has a UV absorption maxima of 205-210 nm, a retention time of approximately 51 minutes on RP-HPLC on a Vydac C₄ column having 5 μm particle size, 330 angstrom pore, 4.6 mm ID×25 cm L in a solvent of 40 mM acetic acid in methanol/water (58/42; v/v) at a flow rate of 1 ml/min, eluting with 69 to 70% methanol from a Vydac C₄ column having 5 μm particle size, 330 angstrom pore, 10 mm ID×25 cm L in a solvent of 40 mM acetic acid with gradient elution from 50 to 80% methanol, with a critical micellar concentration of about 0.03% (w/v) in water and 0.02% (w/v) in phosphate buffered saline, causing hemolysis of sheep red blood cells at concentrations of 25 μg/ml or greater, and containing the monosaccharides terminal rhamnose, terminal arabinose, terminal apiose, terminal xylose, 4-rhamnose, terminal glucose, terminal galactose, 2-fucose, 3-xylose, 3,4-rhamnose, and 2,3-glucuronic acid.

In another embodiment of the invention, the saponin can be in the form of a saponin/antigen conjugate, as described in U.S. Pat. No. 5,583,112, the disclosure of which is incorporated herein by reference in its entirety. In this approach, one or more saponins are linked to an antigen, such that the linkage does not interfere substantially with the ability of the saponin to stimulate an immune response in the animal to which the conjugate is administered.

In another embodiment of the invention, the saponins can be modified to increase their uptake across mucous membranes, for example as described in U.S. Pat. Nos. 5,273,965, 5,443,829 and 5,650,398, the disclosures of which are incorporated herein by reference in their entireties.

In yet another embodiment, the saponins employed in the vaccine compositions of this invention comprise saponin-lipophile conjugates, as described in U.S. Pat. Nos. 5,977,081 and 6,080,725, the disclosures of which are incorporated herein by reference in its entirety. The saponin-lipophile conjugates generally comprise: (1) a non-acylated or deacylated triterpene saponin having a 3-O-glucuronic acid residue, covalently attached to: (2) a lipophilic moiety, for example, one or more fatty acids, fatty amines, aliphatic amines, aliphatic alcohols, aliphatic mercaptans, terpenes or polyethylene glycols; wherein (2) is attached to (1) via the carboxyl carbon atom present on the 3-O-glucuronic acid residue of the triterpene saponin, either directly or through an appropriate linking group.

The attachment of a lipophilic moiety to the 3-O-glucuronic acid of a saponin, such as Quillaja desacylsaponins, Silene jenisseenis, Willd's desacylsaponins, lucyoside P, and Gypsophila Saponaria and Acanthophyllum squarrosum's saponins has been reported to enhance their adjuvant effects on humoral and cell mediated immunity. Additionally, the attachment of a lipophilic moiety to the 3-O-glucuronic acid residue of nonacylated or deacylated saponin may yield a saponin analog that is easier to purify, less toxic and/or chemically more stable, and that may possess equal or better adjuvant properties than the original saponin.

Therefore, the saponins according to this embodiment broadly comprise modified saponins, wherein said modified saponins (a) have a triterpene aglycone core structure (such as quillaic acid, gypsogenin and others) with branched sugar chains attached to positions 3 and 28, and an aldehyde group linked or attached to position 4; (b) are either originally non-acylated, or require removal of an acyl or acyloyl group that is bound to a saccharide at the 28-position of the triterpene aglycone; and (c) have a lipophilic moiety covalently attached, either directly or through a linker moiety, to the carboxylic acid of glucuronic acid at the 3-position of the triterpene aglycone. An example of such a saponin is QS-21 (3):

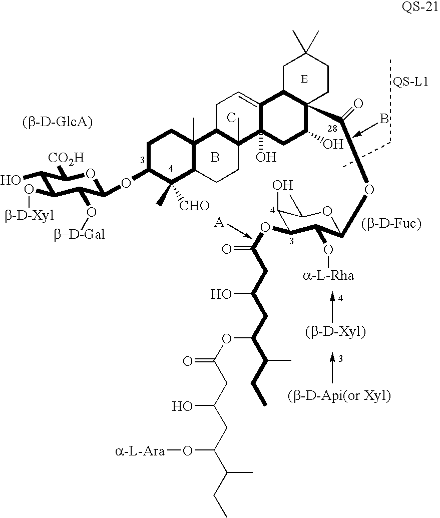

The phrases “lipophilic moiety” and “a residue of a lipophilic molecule,” as used herein, refer to a moiety that is attached by covalent interaction of a suitable functional group of one or more compounds that are non-polar or have a non-polar domain with the 3-O-glcA residue of a saponin. The lipophilic moiety can be a portion of an amphipathic compound. An amphipathic compound is a compound whose molecules contain both polar and non-polar domains. Surfactants are examples of amphipathic compounds. Surfactants typically possess a non-polar portion that is often an alkyl, aryl or terpene structure. In addition, a surfactant possesses a polar portion, that can be anionic, cationic, amphoteric or non-ionic. Examples of anionic groups are carboxylate, phosphate, sulfonate and sulfate. Examples of cationic domains are amine salts and quaternary ammonium salts. Amphoteric surfactants possess both an anionic and cationic domain. Non-ionic domains are typically derivatives of a fatty acid carboxy group and include saccharide and polyoxyethylene derivatives.

A lipophilic moiety can also comprise two or more compounds possessing non-polar domains, wherein each of the compounds has been completely bonded to a linking group, which, in turn, is covalently attached to the 3-O-glucoronic acid.

Several lipophile-containing compounds, such as aliphatic amines and alcohols, fatty acids, polyethylene glycols and terpenes, can be added to the 3-O-glcA residue of deacylsaponins and to the 3-O-glcA residue of non-acylated saponins. The lipophile may be an aliphatic or cyclic structure that can be saturated or unsaturated. By way of example, fatty acids, terpenoids, aliphatic amines, aliphatic alcohols, aliphatic mercaptans, glycosyl-fatty acids, glycolipids, phospholipids and mono- and di-acylglycerols can be covalently attached to nonacylated saponins or desacylsaponins. Attachment can be via a functional group on a lipophilic moiety that covalently reacts with either the acid moiety of the 3-glucuronic acid moiety, or an activated acid functionality at this position. Alternatively, a bifunctional linker can be employed to conjugate the lipophile to the 3-O-glcA residue of the saponin.

Illustrative fatty acids include C₆-C₂₄ fatty acids, preferably C₇-C₁₈ fatty acids. Examples of useful fatty acids include saturated fatty acids such as lauric, myristic, palmitic, stearic, arachidic, behenic, and lignoceric acids; and unsaturated fatty acids, such as palmitoleic, oleic, linoleic, linolenic and arachidonic acids.

Illustrative aliphatic amines, aliphatic alcohols and aliphatic mercaptans include amines and alcohols and mercaptans (RSH) having a straight-chained or branched, saturated or unsaturated aliphatic group having about 6 to about 24 carbon atoms, preferably 6 to 20 carbon atoms, more preferably 6 to 16 carbon atoms, and most preferably 8 to 12 carbon atoms. Examples of useful aliphatic amines include octylamine, nonylamine, decylamine, dodecylamine, hexadecylamine, sphingosine and phytosphingosine. Examples of useful aliphatic alcohols include octanol, nonanol, decanol, dodecanol, hexadecanol, chimyl alcohol and selachyl alcohol.

Illustrative terpenoids include retinol, retinal, bisabolol, citral, citronellal, citronellol and linalool.

Illustrative mono- and di-acylglycerols include mono-, and di-esterified glycerols, wherein the acyl groups include 8 to 20 carbon atoms, preferably 8 to 16 carbon atoms.

Illustrative polyethylene glycols have the formula H—(O—CH₂—CH₂)ₙ—OH, where n, the number of ethylene oxide units, is from 4 to 14. Examples of useful polyethylene glycols include PEG 200 (n=4), PEG 400 (n=8-9), and PEG 600 (n=12-14).

Illustrative polyethylene glycol fatty alcohol ethers, wherein the ethylene oxide units (n) are between 1 to 8, and the alkyl group is from C₆ to C₁₈.

A side-chain with amphipathic characteristics, i.e. asymmetric distribution of hydrophilic and hydrophobic groups, facilitates (a) the formation of micelles as well as an association with antigens, and (b) the accessibility of the triterpene aldehyde to cellular receptors. It is also possible that the presence of a negatively-charged carboxyl group in such a side-chain may contribute to the repulsion of the triterpene groups, thus allowing them a greater degree of rotational freedom. This last factor would increase the accessibility of cellular receptors to the imine-forming carbonyl group.

The desacylsaponins and non-acyl saponins may be directly linked to the lipophilic moiety or may be linked via a linking group. By the term “linking group” is intended one or more bifunctional molecules that can be used to covalently couple the desacylsaponins, non-acylated saponins or mixtures thereof to the lipophilic molecule. The linker group covalently attaches to the carboxylic acid group of the 3-O-glucuronic acid moiety on the triterpene core structure, and to a suitable functional group present on the lipophilic molecule.

Illustrative examples of linker groups which can be used to link the saponin and lipophilic molecule are alkylene diamines (NH₂—CH₂)ₙ—NH₂), where n is from 2 to 12; aminoalcohols (HO—(CH₂)ᵣ—NH₂), where r is from 2 to 12; and amino acids that are optionally carboxy-protected; ethylene and polyethylene glycols (H—(O—CH₂—CH₂)ₙ—OH, where n is 1-4) aminomercaptans and mercaptocarboxylic acids.

In yet another embodiment of the invention, the saponins employed in the compositions of the invention comprise saponin mimetics represented by the following formula (II):

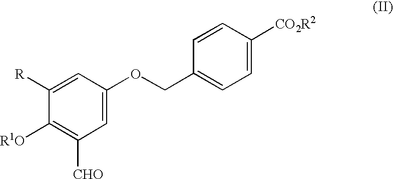

where the symbol R represents hydrogen or —C(O)H. The symbol R¹ represents a member selected from hydrogen, an optionally substituted C₁₋₂₀ aliphatic group, a saccharyl group, and a group represented by the formula —C(O)—[C(R³)(R⁴)]ₖ—COOH or —[C(R³)(R⁴)]ₖ—COOH, wherein each R³ and R⁴ independently is a member selected from hydrogen, a substituted C₁₋₁₀ aliphatic group, or an unsubstituted C₁₋₁₀ aliphatic group. The symbol k represents an integer from 1 to 5. The symbol R² represents a member selected from hydrogen, an optionally substituted C₁₋₂₀ aliphatic group and a group represented by the formula —(CH₂)ᵣCH(OH)(CH₂)ₜOR⁵, wherein r and t are independently 1 or 2, and R⁵ is a C₂₋₂₀ acyl group, or a group represented by the formula

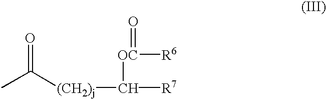

wherein j is an integer from 1 to 5, and R⁶ and R⁷ are independently selected from the group of hydrogen, an optionally substituted C₁₋₂₀ aliphatic group; or a pharmacologically acceptable salt thereof.

In a preferred embodiment, R² is a substituted or unsubstituted aliphatic group having from 1 to 10 carbon atoms, more preferably from 1 to 5 carbon atoms.

In another preferred embodiment, R² is a group represented by the formula: —(CH₂)ᵣCH(OH)(CH₂)ₜOR⁵, in which r and t are independently 1 or 2. The symbol R⁵ is preferably an acyl group having from 2 to 10 carbon atoms, preferably from 10 to 20 carbon atoms.

In another preferred embodiment, R⁵ is a group represented by Formula (III) wherein j is 1, 2, or 3. R⁶ and R⁷ are independently selected from the group of hydrogen and optionally substituted C₁₋₂₀ aliphatic groups.

Although R⁶ and R⁷ can be a branched-, or straight chain, saturated or unsaturated aliphatic group of substantially any length, in a preferred embodiment, R⁶ and R⁷ are each independently aliphatic groups having from 1 to 10 carbon atoms. In a further preferred embodiment, R⁶ and R⁷ are each independently aliphatic groups having from 10 to 20 carbon atoms. In a particularly preferred embodiment, at least one of R⁶ or R⁷ is a substituted or unsubstituted C₁₋₁₁ aliphatic group. In addition to the compounds provided above, the present invention includes pharmacologically acceptable salts of the compounds according to Formula(II).

For those embodiments of compounds of formula (II) in which R¹ is a saccharyl group, a variety of mono-, di-, or polysaccharides are useful. In one preferred embodiment, the saccharyl group is derived from the monosaccharide glucuronic acid, and is selected from either the α- or β- forms of this saccharyl group. As shown below, the site of attachment of the saccharyl group to the remainder of the molecule can be at the reducing end (i.e., the C1 position) of the saccharyl group, as is indicated by the wavy line.

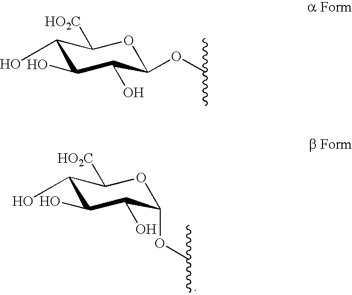

In some embodiments, it is preferred that the saccharyl group is a C₆₋₅₀ saccharyl group, more preferably a C₆₋₃₀ saccharyl group, and still more preferably a C₆₋₂₀ saccharyl group, and yet still more preferably a C₆₋₁₀ saccharyl group.

Within the above general description, a number of embodiments of compounds of formula (II) are particularly preferred. In one preferred embodiment, R, R¹ and R² are all hydrogens, and the compound is isotucaresol, represented by Formula (IV):

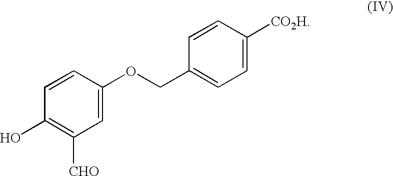

In another preferred embodiment, R is hydrogen, R¹ is a β-D-glucuronic acid group, R² is hydrogen, and the compound is represented by Formula (V):

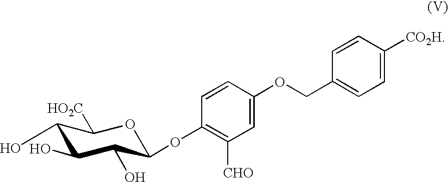

In one embodiment, R is hydrogen, R¹ is a succinoyl group (i.e., R¹=—C(O)—[C(R³)(R⁴)]ₖ—COOH, wherein R³ and R⁴ are hydrogen; k is 2 and R² is hydrogen. The compound is represented by Formula (VI):

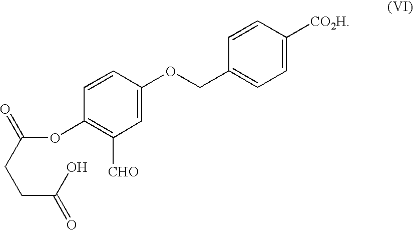

In one embodiment of formula (VI) compounds, R is hydrogen, R¹ is a β-D-glucuronic acid group, and R² is an 1-O-acyl-sn-glyceryl group (sn=stereospecifically numbered; see, Carb. Res. 1998, 312, 167), and the compound is represented by Formula (VII):

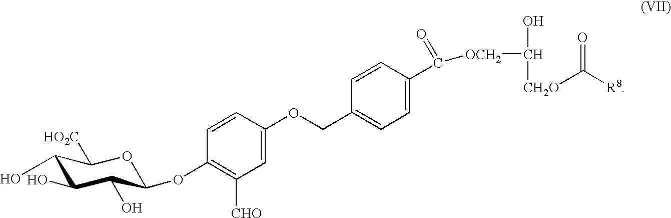

In one embodiment, the acyl group of the 1-O-acyl-sn-glyceryl moiety is acetyl (e.g., R⁸ in Formula VII is methyl; compound 6a), and in another embodiment, octanoyl (R⁸ is heptyl; compound 6b), and in one embodiment, tetradecanoyl (R⁸ is tridecyl; compound 6c).

The amphipathic aldehydes (IV)-(VI) as saponin mimetics possess (are based on?) isotucaresol(IV)as an open-chain analog of quillaic acid (1) which is substituted with lipophilic and/or hydrophilic domains. The design of isotucaresol as a pharmacophore of 1 is based on the premise that saponins are more structurally complex than is necessary for optimal adjuvant effects. Like steroids, the ABC-ring junctures of quillaic acid are all-trans, making the molecule relatively rigid and flat, and thus amenable to molecular mimicry by aromatic seco derivatives. Isotucaresol is an aromatic “triseco” derivative of quillaic acid in which elements of three rings (B, C, E) of the triterpene have been removed but the spatial relationship of key functionality has been maintained.

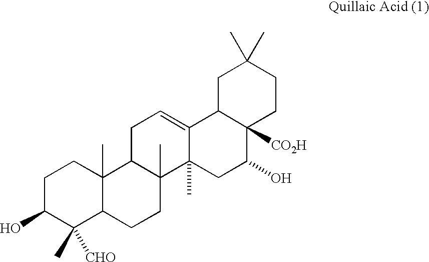

The significance of having two reactive aldehyde moieties on the A-ring of isotucaresol provides the potential for simultaneous engagement of both formyl groups in imine formation with the multiple lysyl E-amino groups (see, Wyss et al., Science 1995, 269: 1273-1278) clustered in the CD2 cell-surface glycoprotein present on T lymphocytes. CD2 is believed to be the principle receptor for Schiff base-mediated costimulation of T-cells (Rhodes, 1996). Multivalent ligand-receptor interactions are common in biological systems and, in the context of T-cell activation, may help to explain not only the immunogenicity of MAA-adducted peptides but also the success of a recent cancer vaccine strategy (see, Apostolopoulos et al., Proc. Natl. Acad. Sci., U.S.A. 1995, 92: 10128-10132) employing formylated mucins.

In another aspect, the present invention includes a compound represented by the Formula II(a):

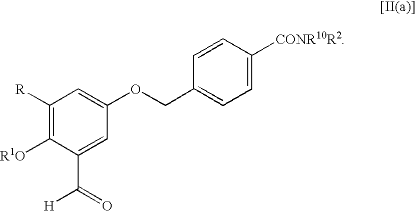

R² and R¹⁰ are independently selected and the symbol R¹⁰ represents a member as described above for R². Compounds of Formula II(a) are useful as adjuvants and immunoeffectors as described herein for compounds of Formulas (Ia)-(Ic).

In another aspect, the present invention provides a compound represented by the Formula II(b):

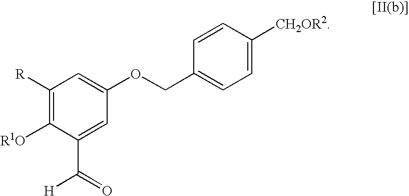

Compounds of Formula II(b) are useful as adjuvants and immunoeffectors as described herein for compounds of Formula (Ia)-(Ic).

By covalently bonding an antigen to an extrinsic adjuvant (immunomodulator) such as a compound of Formula (II, IIa or IIb), a discrete molecule is produced which exhibits a surprisingly unexpected enhanced adjuvanting effect on the antigen which is greater than the adjuvanting effect attainable in the absence of such covalent bonding, as in a mixture of the two components (i.e., the antigen and a compound of Formula (II, IIa or IIb). A further enhanced adjuvanting effect may be attained for such covalently-bonded antigen by incorporating a mineral salt adjuvant with such compounds. The mineral salt adjuvant preferably comprises aluminum hydroxide or aluminum phosphate, although other known mineral salt adjuvants, such as calcium phosphate, zinc hydroxide or calcium hydroxide, may be used.

Aqueous solubility is a desirable characteristic of adjuvant-active saponins and aids in vaccine formulation and efficacy (Kensil, 1996). Unlike oil-based emulsions and mineral salt adjuvants which can denature antigens and prevent protective effects, saponins are non-denaturing adjuvants due to their high aqueous solubility. Their high water solubility also obviates extensive homogenation procedures required for emulsion-type adjuvants, permitting simple mixing of aqueous adjuvant and antigen solutions prior to immunization. Although saponins exhibit a great deal of structural variability in the glycosides attached to C-3 and C-28 of the quillaic acid aglycon unit, the minimal carbohydrate requirement for adjuvanticity (and aqueous solubility) either alone or in formulation (with ISCOMs, alum, etc.) appears to be a glycosidically linked D-glucuronic acid (β-D-GlcA) moiety at C-3 (see, Bomford et al., Vaccine 1992, 10: 572-577; So et al., 1997). Thus, a D-glucuronic acid moiety, glycosidically linked to the phenol group of isotucaresol—itself sparingly soluble at physiologic pH—enhances both aqueous solubility and adjuvanticity, partly by virtue of a second ionizable carboxyl group. Water-soluble O-glycosides of simple hydroxybenzaldehydes (e.g., helicin (31)) not only occur in nature but readily form stable Schiff-base derivatives as well (see, The Merck Index, 12th ed.; Merck & Co., Inc.: Whitehouse Station, NJ, 1996) The synthetically simpler succinate (VI) is also useful since succinic acid constitutes a simple 4-carbon isostere for the glucuronic acid moiety and has been used to impart triterpenes with aqueous solubility (see, Gottfried and Baxendale, U.S. Pat. No. 3,070,623, 1962).

It is important to note that chemical modification of the glucuronic carboxyl of QS-21 does not significantly alter adjuvant activity (Soltysik et al., 1995). Thus, the carboxyl group offers a unique site for attachment of a lipophilic fatty acid domain or a poorly immunogenic peptide. In fact, the attachment of simple lipophilic moieties to the glucuronic acid of deacylated Quillaja saponin or saponins lacking fatty acid domains was recently shown to enhance humoral and cell-mediated immunity (see, Marciani, WO 98/52573, 1998; and U.S. Pat. No. 6,080,725). A peptide determinant linked to the glucuronic carboxyl of a compound of Formula V (or the more lipophilic derivatives according to compounds 6a-6c) would also confer favorable solubility characteristics and potentially provide synthetic vaccines with built-in adjuvanticity. Increased immunogenicity has been observed for lipophilic Quillaja saponins covalently linked to peptide antigens via the glucuronic carboxyl (see, Kensil et al., In Vaccines 92; Brown, F., Chanock, R. M., Ginsberg, H. S., Lerner, R. A., Eds.; Cold Spring Harbor Laboratory Press: Plainview, N.Y., 1992; pp. 35-40).

While not wishing to be bound by the theory or rationale for using hydrophilic Schiff-base-forming compounds lacking fatty acyl groups (i.e., compounds according to Formulae V and VI as adjuvants and immunoeffectors, the use of these compounds deserves further comment. In the case of QS-21 the fatty acid domain, common also to QS-17 and QS-18, plays a critical role: controlled alkaline hydrolysis to give either a desacyl saponin (cleavage at site A in 3) or a quillaic acid derivative (cleavage at the site B) shows that neither of these two hydrolysis products nor the intact fatty acid domain enhance antibody titers or antigen-specific CTLs to ovalbumin when formulated in phosphate buffered saline (PBS) (see, Kensil et al., 1996; Kensil et al., 1992). This and other evidence suggests that antigen binding through hydrophobic interactions is reduced or eliminated when the fatty acid domain is absent. However, a recent study with the QS-21 “B fragment” isolated from unmodified crude Quillaja extract showed that this saponin (designated QS-L1, see, QS-21 partial structure) boosted humoral and cellular immune responses to recombinant hepatitis B surface antigen (rHBsAg) when administered in the presence of alum precipitated antigen. In fact, QS-L1 induced a greater total IgG response in mice than QS-21 to alum-precipitated HBsAg (So et al., 1997). These results suggest the importance of charge interaction between alum, anionic adjuvants, and peptide antigens.

The importance of the fatty acid domain to saponin adjuvanticity is further obscured by the recent structure elucidation of the hydrophilic saponin QS-7 (Kensil et al., 1998). QS-7 is a bisdesmosidic saponin possessing branched sugar units at C-3 and C-28 of quillaic acid similar to those of QS-21, but in contrast possesses an acetyl group in lieu of a large lipid domain on the fucose ring. Like QS-21, QS-7 is a potent inducer of cell-mediated and humoral responses to a variety of antigens, but lacks the characteristic hemolytic activity of saponins towards red blood cells (Kensil, 1996; Kensil et al., 1998). Hemolytic activity—thought to be due to the ability of saponin to intercalate into cell membranes and form a hexagonal array of pores involving cholesterol-complexed saponin molecules -does not correlate with adjuvant activity, however: QS-7 is non-hemolytic whereas digitonin, an adjuvant-inactive steroidal saponin, is highly hemolytic (Kensil, 1996; Kensil et al., 1998; see, Kensil et al., J. Immunol. 1991, 146: 431-437). Thus, CTL induction by exogenous soluble antigen does not appear to be closely associated with either saponin-induced pore formation or the presence of a complex lipophilic domain.

In addition to contributing to the greater toxicity of QS-21 and other lipophilic saponins, the complex fatty acid domain comprising two 3,5-dihydroxy-6-methyl-octanoic acid (DHMO) residues imparts considerable instability to lipophilic saponins. For example, a rapid reversible migration of the DHMO domain occurs between the 3- and 4-hydroxyl groups of fucose in QS-21, confounding purification and purity analysis as well as structure/function assessment (see, Cleland et al., J. Pharm. Sci. 1996, 85: 22-28).

This intramolecular transesterification can be ascribed to the known lability of β-hydroxy esters (see, Sadekov et al., Russ. Chem. Rev. (Eng. Transl.) 1970, 39: 179-195) (to nucleophilic attack by a vicinal hydroxyl in 3, for example). For the same reason, base-catalyzed deacylation is a significant degradation process for QS-21 in aqueous solution, thus limiting the formulations and storage conditions with which QS-21 can be used (Kensil et al., 1995; Cleland, 1996).

Accordingly, the lipophilic derivatives (compounds 6a-c) wherein an sn-glycerol unit (same C-2 relative stereochemistry as D-fucose) has been selected as an open-chain analog of the fucose ring and simple fatty acid residues as stable substitutes for the complex DHMO residues of QS-21; acetate (compound 6a) is an analog of the more hydrophilic and less toxic QS-7. The structural relationship between compounds according to compound 6a and QS-21 is shown in bold in 3.

Combinations of AGPs and Saponins

All types and species of AGPs described herein can be combined with all types and species of saponins described herein for use in the compositions and methods of this invention. Preferred combinations include the AGPs of types (Ia), (Ib) and (Ic), for instance, compounds B3, B9, B14, B15, B19, B22 and B25 mentioned previously, and MPL, with Quillaja saponins such as Quil A and QS-7, -17, -18 or -21, with saponin-lipophile conjugates including GPI-0100, and with tucaresol and other saponin mimetics of formula (II) and their derivatives.

The synthesis of compounds according to Formulae V-VII requires an efficient route to the isotucaresol backbone which is amenable to both scale-up and analog preparation. The original approach to a compound of Formula IV, based on Kneen's multi-step synthesis of tucaresol, (see, Kneen, EP054924, 1986; and U.S. Pat. No. 4,535,183) involved benzofuran starting materials and an ozonolysis step. Other alternate routes for synthesis exist, as discussed below.

Synthesis of Compounds

From a retrosynthetic perspective (Scheme I) the lipophilic isotucaresol compounds can be divided into three major subunits: a glucuronic acid saccharyl unit, an isotucaresol nucleus, and a 3-O-acylated-sn-glycerol unit. Since compounds according Formula V and compounds 6a-c have the glucuronic acid moiety in common, a logical way to assemble these three subunits is by initial glycosylation (or succinoylation in the case of compounds according to Formula VI) of isotucaresol t-butyl ester 7 to give 8, and subsequent selective acylation of the primary hydroxyl group of advanced intermediate 9. This approach reduces the overall number of steps needed to prepare compounds of Formulae V to VII as compared to a divergent strategy involving initial side-chain introduction (compound 6a-c) and permits the potential application of advanced intermediate 9 to the synthesis of other lipophilic derivatives. Further, this route allows incorporation of the chiral synthon 10 late in the synthesis. This synthetic strategy is also suitable for conjugating a peptide to the glucuronic carboxyl with or without a lipophilic side-chain present.

A strategy such as that outlined in Scheme I preferably utilizes orthogonal protection of the aromatic and sugar carboxyl groups as well as protection of the sugar hydroxyl groups of 8 prior to t-butyl ester deprotection and esterification with 10. A t-butyl ester is preferred for benzoate protection due to its stability to the basic conditions of certain o-formylation methods (i.e., 12→7) and its facile acidic cleavage in the presence of the allyl-based protecting groups of the glucuronide. The allyloxycarbonyl (AOC) group is readily introduced into sugars and can be removed along with the allyl ester group under neutral conditions with a palladium (0) catalyst (see, Harada et al., J. Carbohydr. Chem. 1995, 14, 165-170; see, Guibe, Tetrahedron 1998, 54: 2967-3042). Since the Mitsunobu reaction has been used for the stereoselective synthesis of aryl (see, Roush and Lin, J. Am. Chem. Soc. 1995, 117: 2236-2250) and other (see, Smith et al., Tetrahedron Lett. 1986, 27: 5813) β-glycosides from a variety of phenols and sugars including allyl glucuronate 11, (see, Juteau et al., Tetrahedron Lett. 1997, 38: 1481-1484) compound 8 (R=H) can be constructed directly from 7 and 11 using the Mitsunobu protocol. The isotucaresol ring system 7 can also be derived from hydroquinone (13) via benzylation with 14 and o-formylation, or alternatively via a route analogous to Kneen's tucaresol synthesis 22 from benzofuran derivatives 15 and 16.

An alternate approach to the construction of an isotucaresol linchpin, which allows ready access to either (V)-(VII) via a route analogous to Scheme I or compounds 6a-c via a divergent path, o-metalation strategies (see, infra) are also useful for introducing the formyl group. Starting materials which already include an o-formyl group are also useful to prepare compounds of the invention.

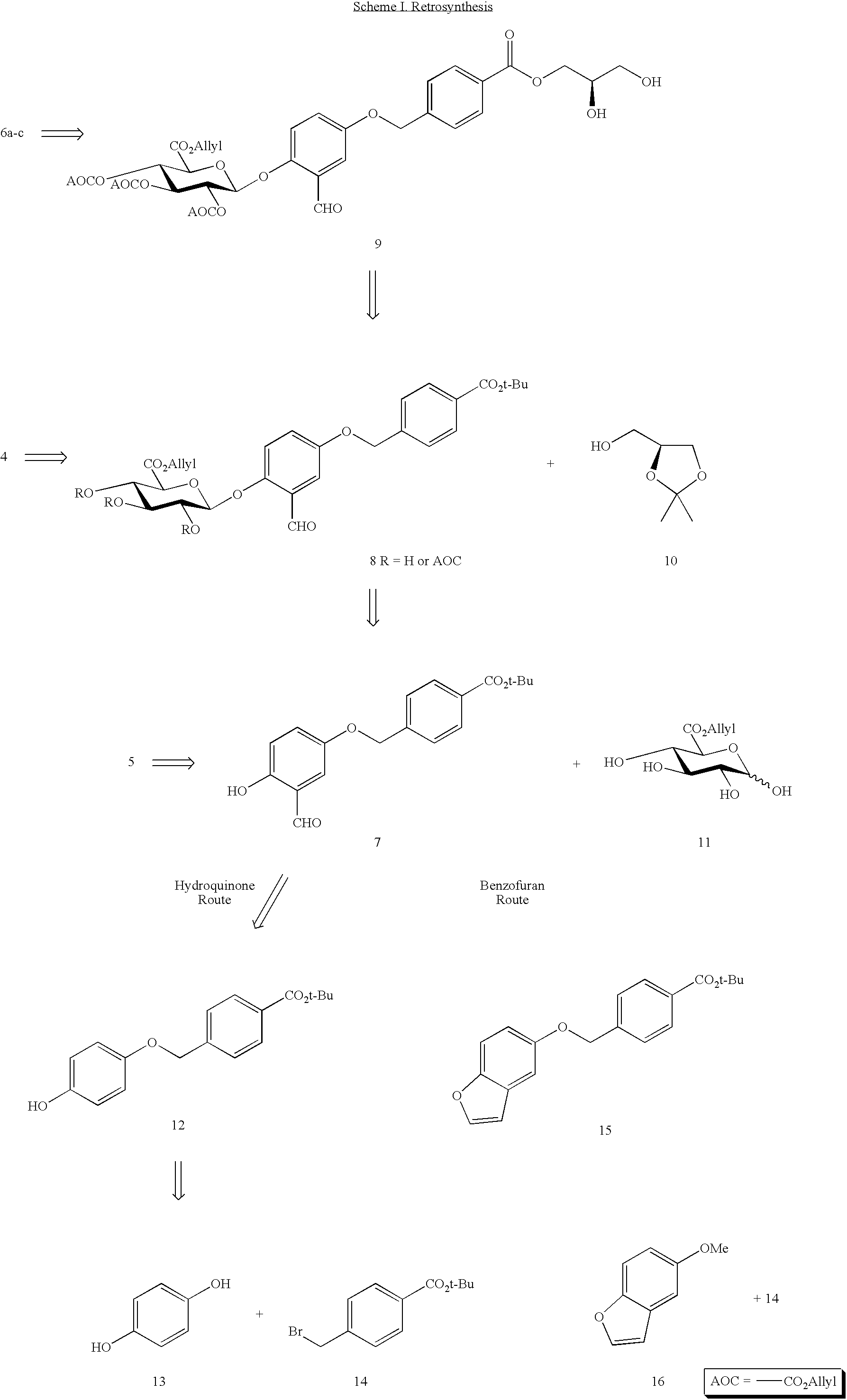

Synthesis of Isotucaresol t-Butyl Ester (7)

Hydroquinone Route

A number of routes are available for constructing t-butyl ester 7, including the o-formylation of phenol 12 (Scheme II). The synthesis of 12 can be readily achieved by monobenzylation of hydroquinone (13) with bromide 14 in the presence of potassium carbonate (see, Schmidhammer and Brossi, J. Org. Chem. 1983, 48:1469-1471) in CHCl₃—MeOH or MeOH or via a recently reported monobenzylating method (see, Zacharie et al., J. Chem. Soc., Perkin Trans. 1 1997, 19: 2925-2930) using Cs₂CO₃ in dimethylformamide (DMF) (The monobenzylation of hydroquinone can also be achieved with free acid 17 under standard conditions (K₂CO₃/MeOH, rt; 60%)). The known t-butyl ester 14 can be prepared according to Zacharie's method (see, Zacharie et al., J. Org. Chem. 1995, 60: 7072-7074) from commercially available 17 with 2-ethoxy-1-ethoxycarbonyl-1,2-dihydroquinoline (EEDQ) and t-BuOH or via one of the other common methods for t-butyl ester formation, such as dicyclohexylcarbodiimide/dimethylaminopyridine (DCC/DMAP) esterification (see, Neises and Steglich, Org. Synth. 1984, 63: 183-187; Greene and Wuts (1991) Protective Groups in Organic Synthesis, 2nd edition, John Wiley & Sons, Inc).

The Reimer-Tiemann reaction can also be used to o-formylate phenols bearing p-substituents (see, Jung and Lazarova, J. Org. Chem. 1997, 62: 1553-1555 and references cited therein.) Thus, treatment of 12 with solid sodium hydroxide and 2 equivalents of water in chloroform at reflux provides isotucaresol t-butyl ester 7 directly.

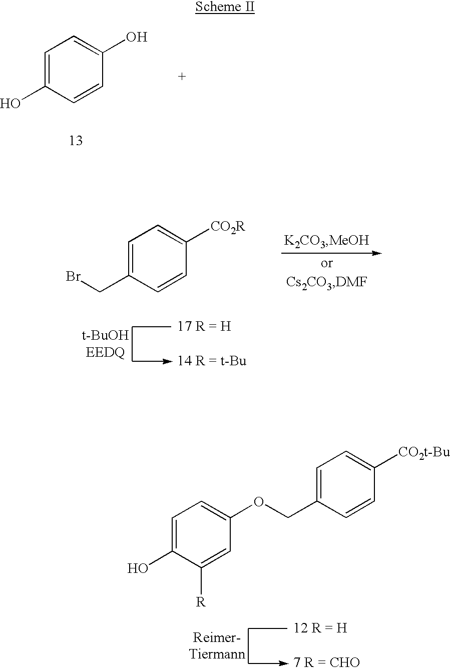

A second method is also available for introducing an o-formyl group into phenol 12 (Scheme III). Recently, Yamaguchi (see, Yamaguchi et al., J. Org. Chem. 1998, 63: 7298-7305) reported that functionalized phenols can be efficiently vinylated at the ortho position with acetylene in the presence of SnCl₄—Bu₃N reagent. Since aryl olefinic groups can be oxidatively cleaved to benzaldehydes in high yield with a variety of reagents (e.g., OsO₄/NaIO₄, RuO₂/NaIO₄) (see, Singh and Samanta, B. Synth. Commun. 1997, 27: 4235-4244; see, Hudlicky, M. Oxidations in Organic Chemistry; Monograph Series 186; American Chemical Society: Washington, D.C., 1990; pp. 77-81)—even in the presence of a free phenolic hydroxyl group, (Singh and Samanta, 1997) the phenol 12 can be converted to salicaldehyde derivative 7 via a two-step process involving stannylacetylene-mediated vinylation of 12 to give 18 and subsequent oxidation with OsO₄/NaIO₄ in aq. dioxane. Alternatively, the crude 18 can be acetylated during work-up—a tactic known to improve vinylphenol stability—and deacetylated (K₂CO₃/MeOH, rt) following oxidation.

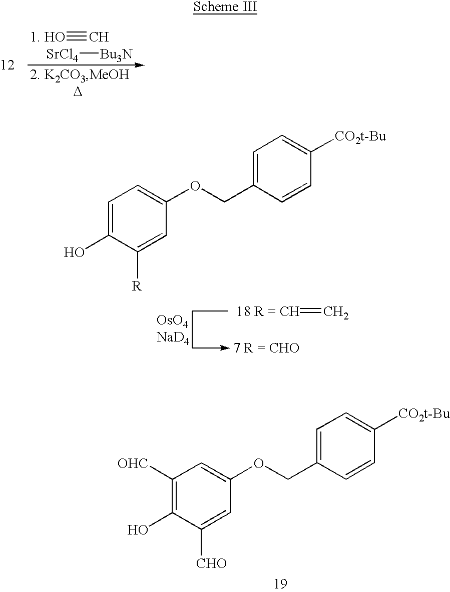

The o-vinylation reaction with acetylene should also allow ready access to the corresponding dicarboxaldehyde 19 and related diformyl derivatives of this invention via divinylation/oxidation of 12 using Yamaguchi's modified reaction conditions for preparing 2,6-divinyl phenols (Yamaguchi et al., 1998). The adjuvant activity of 19 and substituted derivatives can be evaluated using methods described herein.

Directed Metalation Approach to 7

An alternate approach to the o-hydroxybenzaldehyde portion of 7 is the o-metalation of methoxymethyl (MOM)-protected phenol 20 (Scheme IV). The powerful ortho directing ability of the MOM group, coupled with its facile acidic cleavage and base stability, make MOM-ethers especially useful for functionalizing aromatic compounds (see, Zacharie et al., 1997; see, Ronald and Winkle, Tetrahedron 1983, 39: 2031-2042). Thus, hydroquinone 13 can be selectively monoprotected (Zacharie et al., 1997; see, Cruz-Almanza et al., Heterocycles 1994, 37: 759-774) with chloromethyl methyl ether in acetone in the presence of Cs₂CO₃ or via the phenoxide generated with NaH in tetrahydrofuran (THF) to give the known (Cruz-Almanza et al., 1994) MOM-protected phenol 21. Benzylation of 21 with acid 17 in the presence of K₂CO₃ then yields 20. Treatment of 20 with two equivalents of n- or s-butyllithium (RLi) in THF at −78° C. with or without added tetramethylethylenediamine generates the dilithio species, which on quenching at low temperature with DMF yields MOM-protected isotucaresol 22 after aq. NH₄Cl work-up. Directed metalations in the presence of a carboxyl group at low temperature occur without nucleophilic attack (by RLi) on the carboxylate (see, Johnson and Gribble, Tetrahedron Lett. 1987, 28: 5259-5262). It is also possible to convert hydroxy acid 23 directly to 22 by tandem MOM-protection—directed metalation reaction according to the protocol shown in Scheme IV. Similarly, selective methoxymethylation of the dilithio salt of 23 provides an alternate preparation of MOM-ether 20.

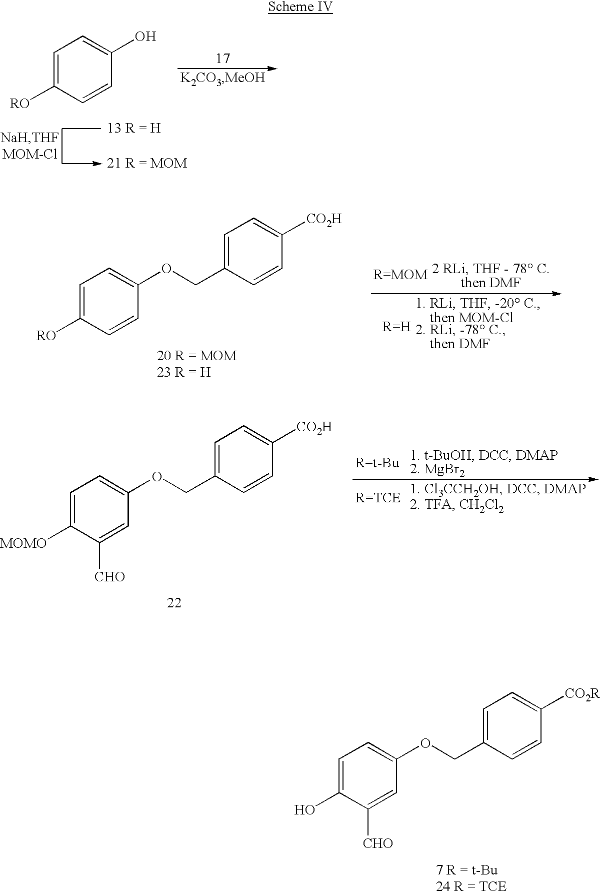

Because compounds 7 and 22 are diametrically protected, 22 is preferred for attaching the lipophilic side-chain first. A compound comprising both lipophilic and hydrophilic domains can be constructed from 22 in as few as 6 steps this way—potentially an important consideration with respect to the large-scale chemical synthesis of an adjuvant candidate.

Compound 22 permits protection of the carboxylic function with groups other than t-butyl since base stability (to phenol o-formylation) is obviated. Selective deprotection of the MOM group in the presence of a t-butyl ester is possible with reagents such as B-bromocatecholborane or MgBr₂, where removal is facilitated by chelation with the neighboring carbonyl (see, Haraldsson and Baldwin, Tetrahedron. 1997, 53: 215-224). Alternatively, initial deprotection of the MOM group and selective t-butyl ester formation using in situ-generated isobutylene (see, Wright et al., Tetrahedron Lett. 1997, 38: 7345-7348) can be used to provide intermediate 7. Accordingly, 22 is converted to 7 by one these two protocols or, alternatively, to the 2,2,2-trichloroethyl (TCE) ester 24 by carbodiimide esterification and MOM removal with TFA, etc. TCE esters are stable to a greater range of glycosylating conditions than t-butyl esters, but like t-butyl groups are orthogonal to allyl-based sugar protection (see, Greene and Wuts, Protective Groups in Organic Synthesis; 2nd ed.: John Wiley & Sons, Inc.: New York, 1991; pp. 240-241).

Synthesis of Compound 7 from 2,5-Dihydroxybenzaldehyde (25)

One variation on the hydroquinone strategy, which—like the benzofuran route below—commences with a fully functionalized A-ring, is the selective benzylation of 2,5-dihydroxybenzaldehyde (25) on the more nucleophilic 5-hydroxyl group. Thus, treatment of commercially available 25 with bromide 14 under conditions known to selectively alkylate the hydroxyl meta to the carbonyl group in 2,5-dihydroxy systems (see, Sadekov et al., 1970; see, Vyas and Shah, Org. Synth., Coll. Vol. 4 1963, pp. 836-839) can be used to provide intermediate 7 in just two steps (Scheme V). Likewise, alkylation of 25 with acid 17 gives isotucaresol (IV) in a single step.

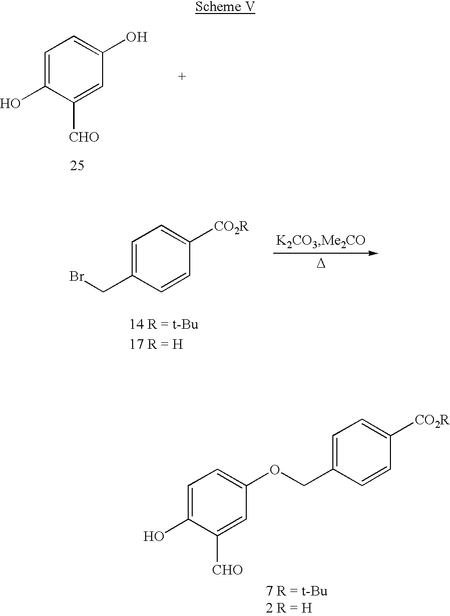

Selective debenzylation ortho to an aromatic carbonyl group to yield Comopund 7 or (IV)

Compounds according to the formulae of 7 or IV can be made by selective debenzylation. For example, a dibenzylated product formed as a side product in the preparation of 7 (or IV) in Scheme V can be selectively cleaved at the site ortho to the formyl group with MgBr₂ (see, Haraldsson and Baldwin, 1997). Alternatively, quantitative dibenzylation of 25 with 14 or 17, or other appropriate derivatives followed by selective o-debenzylation also provides an efficient route to (IV) and its derivatives (e.g., compound 40). The simplicity of these methods offsets the greater expense of the starting material 25 as compared to hydroquinone 13.

Generally, this reaction scheme is carried out on in the presence of a Lewis acid to form the selectively debenzylated product as in Scheme VI:

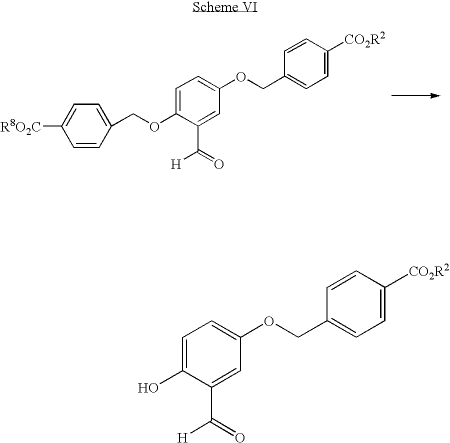

R² and R⁸ can be the same or different. In some embodiments, R² and R⁸ are selected from moieties which are known in the art as carboxylic acid protecting groups. Compounds within the scope of the invention include embodiments where R² and R⁸ are independently selected from hydrogen, a substituted C₁₋₂₀ alkyl group, an unsubstituted C₁₋₂₀ alkyl group, and a group having the formula —(CH₂)_{rCH}(OH)(CH₂)ₜOR⁵ wherein r and t are independently 1 or 2, and R⁵ is a substituted C₂₋₂₀ acyl group or a group having the formula:

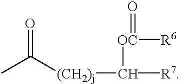

The symbol j represents an integer from 1 to 5. The substituents R⁶ and R⁷ can independently represent hydrogen, a substituted C₁₋₂₀ alkyl group, or an unsubstituted C₁₋₂₀ alkyl group.

The o-debenzylation can be achieved with a Lewis acid having the formula MXₙ. M is selected from the group containing Al³⁺, As³⁺, B³⁺, Fe²⁺, Fe³⁺, Ga³⁺, Mg²⁺, Sb³⁺, Sb⁵⁺, Sn²⁺, Sn⁴⁺, Ti²⁺, Ti³⁺, Ti⁴⁺, and Zn²⁺. X is a halide selected from Cl, I, F, and Br. Those of skill in the art will recognize that n is an integer from 2 to 5 depending on the valence state of M. In some embodiments, the Lewis acids that can be used to achieve the ortho-debenzylation include, but are not limited to: AlCl₃, AlI₃, AlF₃, AlBr₃, Et₂AlCl, EtAlCl₂, AsCl₃, AsI₃, AsF₃, AsBr₃, BCl₃, BBr₃, BI₃, BF₃, BCl₃.SMe₂, BI₃.SMe₂, BF₃.SMe₂, BBr₃.SMe₂, FeCl₃, FeBr₃, FeI₃, FeF₃, FeCl₂, FeBr₂, FeI₂, FeF₂, GaCl₃, GaI₃, GaF₃, GaBr₃, MgCl₂, MgI₂, MgF₂, MgBr₂, MgCl₂—OEt₂, MgI₂—OEt₂ MgF₂—OEt₂ MgBr₂—OEt_{2, SbCl}₃, SbI₃, SbF₃, SbBr₃, SbCl₅, SbI₅, SbF₅, SbBr₅, SnCl₂, SnI₂, SnF₂, SnBr₂, SnCl₄, SnI₄, SnF₄, SnBr₄, TiBr₄, TiCl₂, TiCl₃, TiCl₄, TiF₃, TiF₄, TiI₄, ZnCl₂, ZnI₂, ZnF₂, and ZnBr₂. In addition, the o-debenzylation can be achieved with Lewis acids such as Et₂AlCl, EtAlCl₂, monoalkyl boronhalides, dialkyl boronhalides, and monoaryl boronhalides, diaryl boronhalides. X can be, but is not limited to, Cl, I, F, and Br. The reaction is carried out under conditions sufficient to form the ortho-debenzylated product. These conditions can be determined by one of skill in the art by optimizing reaction parameters. Reaction parameters that can be optimized in the ortho-debenzylation reaction include, but are not limited to, length of reaction incubation, temperature, pressure, solvent(s), ratio of solvent to starting materials, etc. Methods of optimizing reactions of the present invention are well within the purview of one skilled in the organic chemistry arts.

Without being bound by any particular theory, the reaction of these Lewis Acids with the dibenzylated starting material is thought to form a multi-membered (e.g., six-membered) chelation ring intermediate. This multi-membered chelation ring intermediate is then subjected to hydrolysis (e.g., with a base, an acid, HCl, etc.) to yield the ortho-debenzylated product. The addition of a base or acid to the reaction mixture can be considered part of the conditions sufficient to form the desired ortho-debenzylated product.

In some embodiments, the o-debenzylation is carried out by reacting compound 39 under condition A, condition B, or condition C to give methyl 4-(3-formyl-4-hydroxyphenoxymethyl)benzoate (isotucaresol methyl ester; 40):

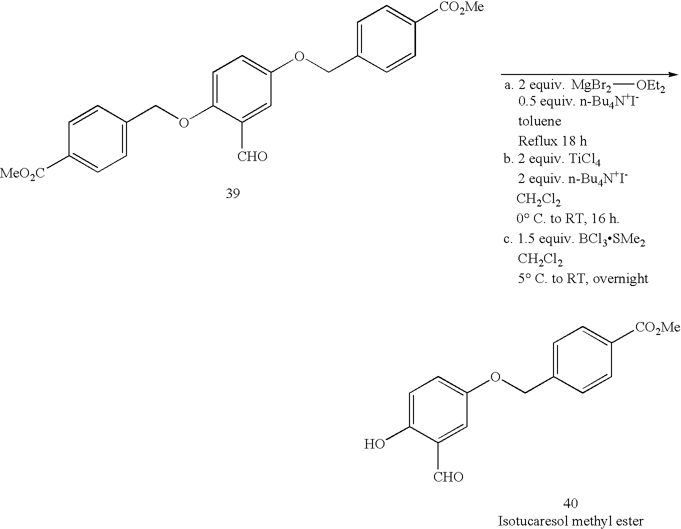

Benzofuran Route

In one synthesis of isotucaresol (IV), commercially available 5-methoxybenzofuran (16) is demethylated with boron tribromide (see, Williard and Fryhle, Tetrahedron Lett. 1980, 21: 3731-3734) to give 26, which is then benzylated with methyl 4-(bromomethyl)benzoate. Analogous benzylation of 26 with t-butyl ester 14 and ozonolysis (Kneen, EP054924, 1986; and U.S. Pat. No. 4,535,183) of benzofuran intermediate 15 provides compound 7 (Scheme VII).

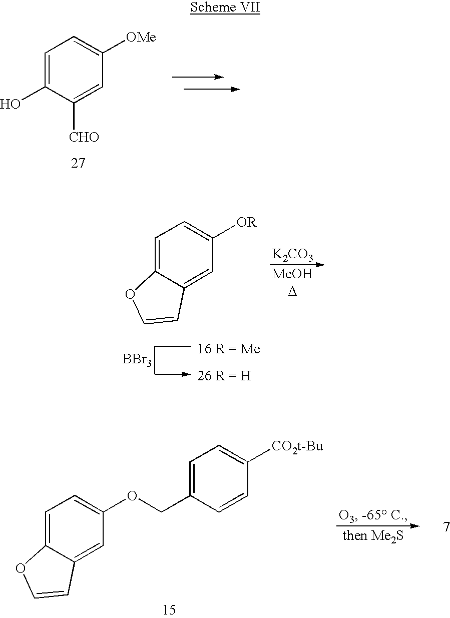

Synthesis of Hemisuccinate (V)

Conversion of phenols and alcohols to their corresponding hemisuccinates (isolated as the free acid or alkali metal salt) is a common tactic to enhance aqueous solubility of steroids and other lipophilic drugs, and consequently general methods are available for succinoylation (see, Gottfried and Baxendale, 1962). Treatment of t-butyl ester 7 with succinic anhydride in pyridine yields compound (V) subsequent to deprotection of the t-butyl ester with trifluoroacetic acid (TFA) (Scheme VIII). Since quaternary carboxylic acid groups do not ordinarily interfere with this reaction, the direct succinoylation of isotucaresol (IV) is also possible.

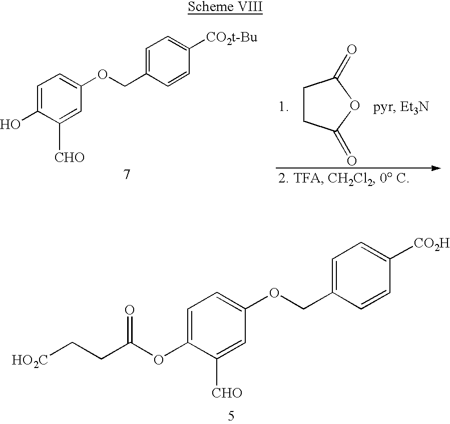

Synthesis of Glucuronide 4

The highly stereoselective synthesis of aryl β-glycosides and acyl β-glucuronides has been achieved via the Mitsunobu reaction (see, Roush and Lin, 1995; see, Smith et al., 1986). In fact, allyl glucuronate 11 has been used in the Mitsunobu reaction without protection of sugar hydroxyl groups in yields up to 50% by taking advantage of the higher reactivity of the anomeric hydroxyl group (see, Juteau et al., 1997). Application of the Mitsunobu protocol to fully protected sugars gives even higher yields (70-95%) of aryl β-glycosides (see, Roush and Lin, 1995).

Accordingly, the known (Juteau et al., 1997) allyl ester 11, prepared from D-glucuronic acid and allyl bromide (1,8-diazobicyclo[5.4.0]undec-7-ene (DBU)/DMF, rt) in 75% yield, is selectively coupled with phenol 7 or a related derivative in the presence of triphenylphosphine and diisopropylazodicarboxylate (DIAD) in THF at 0° C. to give aryl β-glycoside 28 (i.e., 8 R=H) as shown in Scheme IX. Sequential deprotection of the ester protecting groups with TFA and Pd(0) in the presence of a suitable allyl scavenger (see, Harada et al., 1995; see, Guibe, 1998) then gives compound (V).

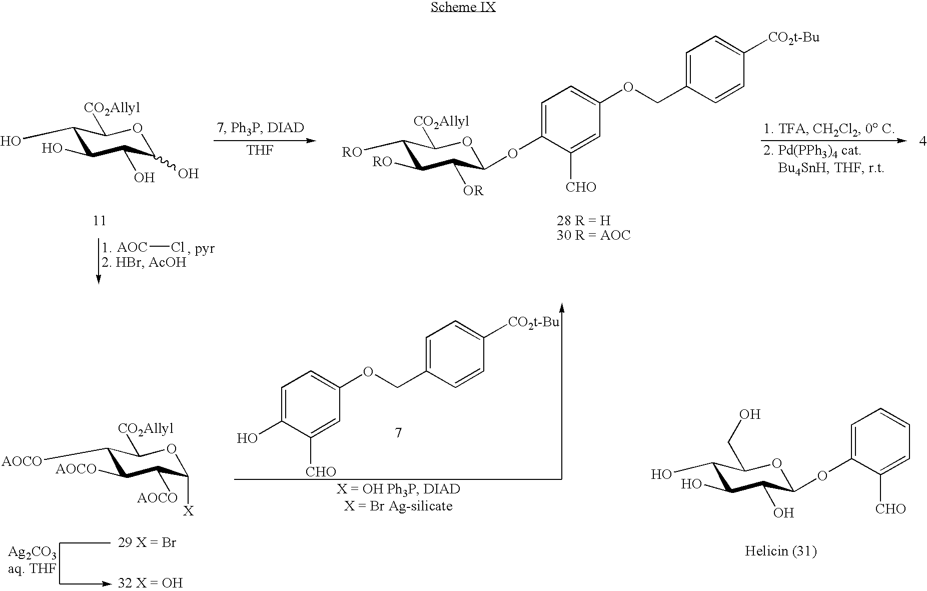

An alternate method which has been used for the glycosylation of phenols is the Koenigs Knorr reaction of pyranosyl bromides in the presence of a silver salt (see, Roush and Lin, 1995; see, Robertson and Waters, R.B.J. Chem. Soc. 1930, 2729-2733) Since AOC groups have been introduced onto the 2,3,4-positions of glucuronides in high yield using AOC-Cl in pyridine, (see, Harada et al., 1995) 11 is similarly protected and then treated with HBr in acetic acid to give bromide 29. Silver mediated coupling of 29 and 7 then gives predominantly the aryl β-glycoside 30 (i.e., 8, R=AOC). An analogous glycosylation has been used to prepare the natural product helicin (31) from salicaldehyde and O-tetraacetyl-4-D-glucopyranosyl bromide in the presence of silver oxide (see, Robertson and Waters, 1930). Glycosyl donor 29 also provides access to lactol 32 by silver-mediated hydrolysis (see, Roush and Lin, 1995). Mitsunobu reaction of fully protected 32 with 7 should also give 30, which can then be deprotected to 4 by the same 2-step deprotection as for 28.

Synthesis of Glucuronides 6a-6c

Aryl glycoside 30, prepared directly from 29 or 32 as discussed above or, alternatively, by AOC-protection of 28, is transformed into the advanced intermediate 9 by the sequence: (1) t butyl ester hydrolysis, (2) esterification with 10, and (3) acetonide cleavage as shown in Scheme X below. Recently, in an approach to aureolic acid antibiotics it was demonstrated that aryl glycosides possessing electron-withdrawing substituents on the aromatic aglycon are stable to acidic deprotection of ketal and other protecting groups (Roush and Lin, 1995; Roush et al., J. Am. Chem. Soc. 1999, 121: 1990-1991). In fact, certain phenyl glycosides bearing carbonyl groups in the aglycon unit have shown remarkable stability to acidic hydrolysis (see, Bär et al., Wiss. Technol. 1990, 23: 371-376). Nevertheless, if the glycosidic linkage is sensitive to ketal and/or t-butyl ester cleavage, TCE ester 24—prepared from 22 or via ester interchange of 7—can be used for glucuronidation and subsequently deprotected under neutral conditions with zinc in buffered aq. THF (see, Just and Grozinger, Synthesis 1976, 457-458). Stable isosteres (pseudosugar, C-glycoside) of the glucuronide can be prepared.

Compound 9 is selectively acylated on the primary hydroxyl group with acetic anhydride and the appropriate acid chlorides under standard conditions to give 33a-c. Although acetylations with acetyl chloride are not as selective as with other acid chlorides, acetyl introduction with Ac₂O in CHCl₃ in the presence of pyridine provides good selectivity for primary alcohols when the reaction is run below 0° C. (see, Stork et al., J. Am. Chem. Soc. 1978, 100: 8272-8273). One method that has been applied specifically to the selective acylation of glycerol derivatives is the reaction of an in situ-generated stannoxane—prepared with Bu₂SnO in toluene by azeotropic dehydration—with acid chlorides at 0° C. (see, Aragozzini et al., Synthesis 1989, 225-227). Deprotection of the allyl-based protecting groups of 33a prepared by one of these methods delivers (6a-c).

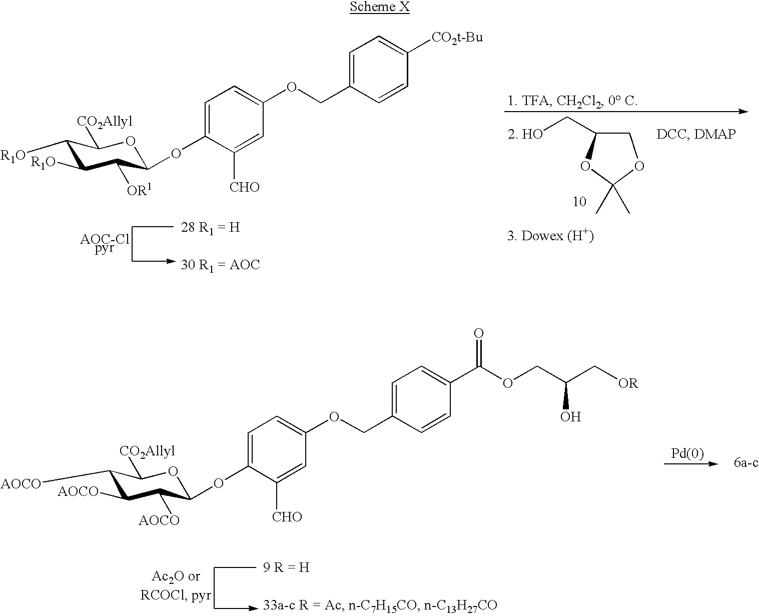

Divergent Synthesis of 6a-c

As discussed above, MOM ether 23 is ideally suited for elaborating the acylated glycerol unit prior to glucuronidation of the phenolic hydroxyl group. Thus, esterification of 23 with 10, followed by acetonide hydrolysis and acylation as described above should yield 34a-c (Scheme XI). MOM deprotection and Mitsunobu coupling of the resulting 35a-c with 11 then provides glucuronides 36a-c, which can be deprotected with Pd(0) to give 6a-c.

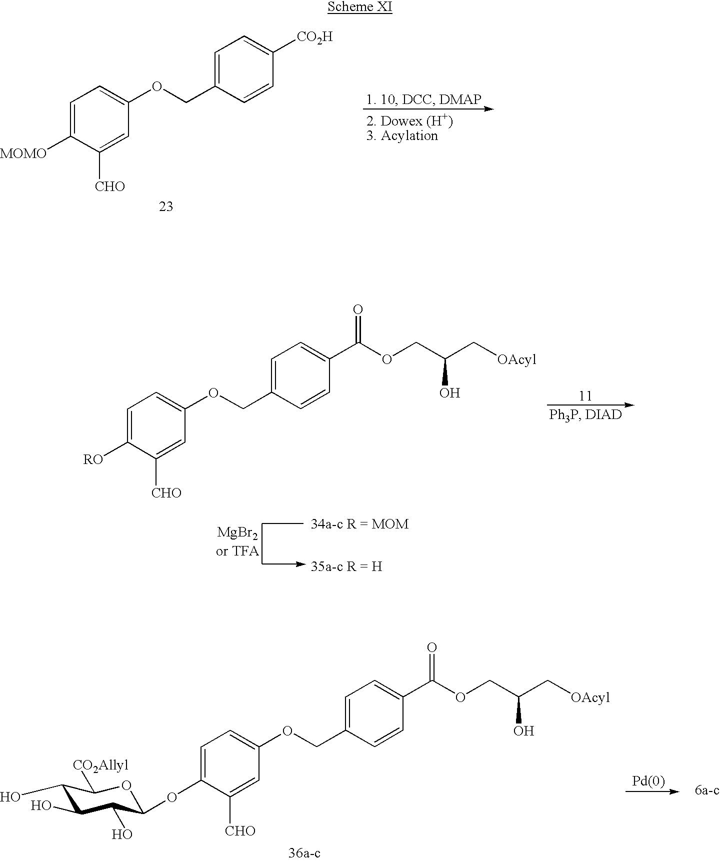

Finished products (IV-VI) are analyzed by standard spectroscopic (IR, ¹H and ¹³C NMR) and physical (elemental and HRMS) data. Purity is assessed by reverse-phase HPLC analysis of the intact molecules or a suitable derivative (e.g., phenacyl ester of the glucuronic carboxyl group).

### IV. Evaluation of Compounds

The adjuvant effects of a composition containing an AGP compound and a saponin on humoral and cell-mediated responses can be determined in two different murine models using rHBsAg (recombinant Hepatitis B Surface Antigen), inactivated influenza virus (e.g., hemagglutinin protein in FluZone influenza vaccine (Connaught Laboratories, Swiftwater, Pa.)) as antigens (see also Example section below). In the case of rHBsAg, the compounds can be formulated with both alum-adsorbed antigen and soluble antigen and compared with an alum-adsorbed antigen control. Antibody titers (e.g., IgG, IgG1, IgG2a, IgG2b, etc.) to rHBsAg can be determined by ELISA from pre-vaccination and post-vaccination sera.

Given the enhanced serum and mucosal CTL and IgA responses often elicited with vaccines administered intranasally (i.n.), (see, VanCott et al., J. Immunol. 1998, 160: 2000-2012; Imaoka et al., J. Immunol. 1998, 161: 5952-5958) both i.n. and subcutaneous (s.c.) immunization of mice are performed with the above formulations. The compounds are evaluated for their ability to induce rHBsAg-specific antibodies and influenza hemagglutinin-specific antibodies in BALB/c mice and enhance CTLs against P815S-HBsAg target cells (see, e.g., Moore et al., (1988) Cell 55: 777-785). The P815S cell line is a transfectant of P815 which expresses the HBsAg CTL_{S28-39} epitope in the MHC-I complex and shows relevance to human immune responses to hepatitis B virus (HBV), for which CTL responses appear to be important for pathogen clearance (see, Schirmbeck et al., J. Immunol. 1994, 152: 1110-1119; see, Schirmbeck et al., J. Virol. 1994, 68: 1418-1425).

### V. Pharmaceutical Compositions

Formulations

The combination of an AGP (e.g., a compound of Formula Ia, Ib or Ic) and a saponin (e.g., a compound of formula II, IIa, IIb, QS-21, etc.) can be formulated with a pharmaceutically acceptable carrier for administration to a subject. While any suitable carrier known to those of ordinary skill in the art may be employed in the pharmaceutical compositions of this invention, the type of carrier will vary depending on the mode of administration. The pharmaceutical composition is typically formulated such that the AGP and the saponin are present in a combined effective immunopotentiatory amount or a therapeutically effective amount, i.e., the amount of compound required to achieve the desired effect in terms of treating a disease or condition, or achieving a biological occurrence. In one embodiment of the invention, each of the AGP and saponin are present in amounts that, individually, provide therapeutic effects, and the overall effect of the combination is a synergistic one, i.e., provides a combination effect that exceeds any expected additive effect. However, since the compositions of this invention include those having a combination synergistic effect, they include compositions and methods in which one, or even both, of the AGP and saponin are provided in amounts that individually are less than those needed to provide a therapeutic effect; however, the combination, surprisingly, is therapeutically effective.

In one embodiment, the effective amount of the an AGP and saponin ranges from 0.0001 to about 1.0 mg/kg of body weight of the subject mammal, more preferably from 0.001 to about 0.1 mg/kg of body weight of the mammal. In one embodiment, an AGP and a saponin are administered once weekly to once monthly for a period of up to 6 months, more preferably once monthly for a period of about 2-3 months. In one aspect, the present invention provides a method of treating or preventing a disease in a mammal comprising administering to said mammal a vaccine composition comprising an antigen and an effective immunopotentiatory amount of an AGP and a saponin. The diseases include cancer, autoimmune disease, allergy and infectious disease (such as bacterial and viral infection).

The AGPs and saponins used in the pharmaceutical compositions of this invention have a wide range of activities. Some AGPs are much more active than others, and similarly, some saponins are much more active than others. The choice of a particular AGP and a particular saponin for use in a given situation will in general be based on a number of factors, with the pharmaceutical activities being only one. In a given case it may be desirable to use a combination of an AGP having a relatively high activity with a saponin having only a relatively moderate level of activity. Accordingly, compositions containing combinations of different AGPs and/or saponins, respectively, are likely to contain different amounts of these two materials Liquid compositions for direct administration to a patient (i.e., single-dosage formulations) will in general contain from about 100 μg/mL to about 10 mg/mL. The amount of the AGP administered to the patient will range from about 1 nanogram to about 1 millgram per kg body weight, preferably from about 10 nanograms to about 100 micrograms. The amount of a saponin administered to a patient will generally range from about 100 ng to about 10 mg per kg body weight, preferably from about 1 μg to about 5 mg. Dry formulations or more concentated liquid formulations of AGPs with saponins will contain those amounts of materials that, when diluted or otherwise adjusted, will provide the above dosages.

Similarly, because combinations of AGPs and saponins of comparatively different levels of activity may be used together in a given composition or formulation, the weight ratio of AGP to saponin in the compositions of this invention can vary over a wide range. In general, the two ingredients are present in the compositions in a weight ratio of AGP to saponin of from about 1:1000 to about 1000:1, preferably from about 100:1 to about 1:100. Preferably they are in such a weight ratio that the unexpected or synergistic effects of using the combination of AGP and saponin are achieved.

Preferred compositions thus are those in which the AGP and saponin are present in synergistically effective amounts, i.e. amounts which, when the composition is administered to a subject, have a synergistic or other unexpected effect as compared to the use of the individual AP and saponin alone. Similarly, methods of using the compositions preferably include the administration of a composition containing an AGP and a saponin such that the composition provides a synergistic or other unexpected effect as compared to the administration of the AGP or saponin alone.

It should be noted that while the compositions and methods of administration described herein are depicted in terms of containing one AGP and one saponin, this terminology is used merely as a convenience. Compositions and methods of use according to this invention may in fact contain one or more than one AGP and/or one or more than one saponin, as may be appropriate for a given treatment. The amounts and proportions of AGP and saponin provided herein refer to the total content or ratio of the respective category of ingredient.

For preparing pharmaceutical compositions, the pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

Solid forms of the compositions can be prepared, for instance, by spray-drying aqueous formulations of the active adjuvants (e.g. in the form of a salt) or by lyophilizing them and milling with excipients.

Suitable carriers for the solid compositions of this invention include, for instance, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. In certain embodiments, the pharmaceutical compositions are formulated in a stable emulsion formulation (e.g., a water-in-oil emulsion or an oil-in-water emulsion)or an aqueous formulation that preferably comprise one or more surfactants. Suitable surfactants well known to those skilled in the art may be used in such emulsions. In one embodiment, the composition comprising the AGP and the saponin is in the form of a micellar dispersion comprising at least one suitable surfactant. The surfactants useful in such micellar dispersions include phospholipids. Examples of phospholipids include: diacyl phosphatidyl glycerols, such as: dimyristoyl phosphatidyl glycerol (DPMG), dipalmitoyl phosphatidyl glycerol (DPPG), and distearoyl phosphatidyl glycerol (DSPG); diacyl phosphatidyl cholines, such as: dimyristoyl phosphatidylcholine (DPMC), dipalmitoyl phosphatidylcholine (DPPC), and distearoyl phosphatidylcholine (DSPC); diacyl phosphatidic acids, such as: dimyristoyl phosphatidic acid (DPMA), dipalmitoyl phosphatidic acid (DPPA), and distearoyl phosphatidic acid (DSPA); and diacyl phosphatidyl ethanolamines such as: dimyristoyl phosphatidyl ethanolamine (DPME), dipalmitoyl phosphatidyl ethanolamine (DPPE), and distearoyl phosphatidyl ethanolamine (DSPE). Other examples include, but are not limited to, derivatives of ethanolamine (such as phosphatidyl ethanolamine, as mentioned above, or cephalin), serine (such as phosphatidyl serine) and 3′-O-lysyl glycerol (such as 3′-O-lysyl-phosphatidylglycerol).

Typically, a surfactant:adjuvant molar ratio in an aqueous formulation will be from about 10:1 to about 1:10, more typically from about 5:1 to about 1:5, however any effective amount of surfactant may be used in an aqueous formulation to best suit the specific objectives of interest.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The pharmaceutical compositions can be adminstered as an immunostimulant composition in the absence of an antigen. Such compositions can be used to treat a subject (e.g., a mammal) suffering from or susceptible to a pathogenic infection, cancer or an autoimmune disorder. In other embodiments, the compositions can be adminstered to enhance immune response in an animal.

Antigens and Vaccine Formulations

In other embodiments, the immune response of an animal (e.g., a human) can be enhanced by adminstering the composition in combination with an antigen; the adjuvant system of the present invention can be administered without a co-administered antigen, to potentiate the immune system for treatment of chronic infectious diseases, especially in immune compromised patients. Illustrative examples of infectious diseases for which this approach may be employed for therapeutic or prophylactic treatment can be found in U.S. Pat. No. 5,508,310. Potentiation of the immune system in this way can also be useful as a preventative measure to limit the risks of nosocomial and/or post-surgery infections.

The pharmaceutical compositions can act as an adjuvant when co-administered with an antigen. The compounds of Formulae I(a-c), II, III, IV, IVa, and IVb, and the other saponins and AGPs set out herein can be thought of as the extrinsic adjuvant. An adjuvant is an immunostiumulatory agent that enhance the immunogenicity of an antigen but is not necessarily immunogenic itself. Intrinsic adjuvants, such as lipopolysaccharides, normally are the components of the killed or attenuated bacteria used as vaccines. Extrinsic adjuvants are immunomodulators which are typically non-covalently linked to antigens and are formulated to enhance the host immune responses. In one embodiment, the antigen is a tumor associated antigen (tumor specific antigen).

In one embodiment the present invention provides a vaccine composition comprising an antigen and a saponin and an AGP. Suitable antigens include microbial pathogens, bacteria, viruses, proteins, glycoproteins lipoproteins, peptides, glycopeptides, lipopeptides, toxoids, carbohydrates, and tumor-specific antigens. Mixtures of two or more antigens may be employed.

Thus, the adjuvant systems of the invention are particularly advantageous in making and using vaccine and other immunostimulant compositions to treat or prevent diseases, such inducing active immunity towards antigens in mammals, preferably in humans. Vaccine preparation is a well developed art and general guidance in the preparation and formulation of vaccines is readily available from any of a variety of sources. One such example is New Trends and Developments in Vaccines, edited by Voller et al., University Park Press, Baltimore, Md., U.S.A. 1978.

The vaccine compositions of the present invention may also contain other compounds, which may be biologically active or inactive. For example, one or more immunogenic portions of other tumor antigens may be present, either incorporated into a fusion polypeptide or as a separate compound, within the vaccine composition. Polypeptides may, but need not, be conjugated to other macromolecules as described, for example, within U.S. Pat. Nos. 4,372,945 and 4,474,757. Vaccine compositions may generally be used for prophylactic and therapeutic purposes.

In one illustrative embodiment, the antigen in a vaccine composition of the invention is a peptide, polypeptide, or immunogenic portion thereof. An “immunogenic portion,” as used herein is a portion of a protein that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of an antigenic protein or a variant thereof.

Immunogenic portions of antigen polypeptides may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well known techniques. An immunogenic portion of a protein is a portion that reacts with such antisera and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Peptide and polypeptide antigens are prepared using any of a variety of well-known techniques. Recombinant polypeptides encoded by DNA sequences may be readily prepared from isolated DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO.

Portions and other variants of a protein antigen having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See, Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide antigen used in the vaccine compositions of the invention may be a fusion protein that comprises two or more distinct polypeptides. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

Within preferred embodiments, an immunological fusion partner is derived from protein D, a surface protein of the gram-negative bacterium Haemophilus influenza B (See, e.g., WO 91/18926, U.S. Pat. Nos. 6,139,846, 6,025,484, 5,989,828, 5,888,517, and 5,858,677). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemagglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see, Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In another embodiment of the invention, the adjuvant system described herein is used in the preparation of DNA-based vaccine compositions. Illustrative vaccines of this type contain DNA encoding one or more polypeptide antigens, such that the antigen is generated in situ. The DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In one preferred embodiment, the DNA is introduced using a viral expression system (e.g., vaccinia or other pox virus, retrovirus, or adenovirus), which typically involves the use of a non-pathogenic (defective), replication competent virus. Illustrative systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art.

Alternatively, the DNA may be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads that are efficiently transported into the cells. It will be apparent that a vaccine may comprise both a polynucleotide and a polypeptide component if desired.

Moreover, it will be apparent that a vaccine may contain pharmaceutically acceptable salts of the desired polynucleotide, polypeptide and/or carbohydrate antigens. For example, such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

The adjuvant system of the present invention exhibits strong adjuvant effects when administered over a wide range of dosages and a wide range of ratios.

The amount of antigen in each vaccine dose is generally selected as an amount which induces an immunoprotective response without significant adverse side effects in typical vaccines. Such amount will vary depending upon which specific immunogen is employed and how it is presented. Generally, it is expected that each dose will comprise about 1-1000 μg of protein, most typically about 2-100 μg, preferably about 5-50 μg. Of course, the dosage administered may be dependent upon the age, weight, kind of concurrent treatment, if any, and nature of the antigen administered.

The immunogenic activity of a given amount of a vaccine composition of the present invention can be readily determined, for example by monitoring the increase in titer of antibody against the antigen used in the vaccine composition (Dalsgaard, K. Acta Veterinia Scandinavica 69:1-40 (1978)). Another common method involves injecting CD-1 mice intradermally with various amounts of a vaccine composition, later harvesting sera from the mice and testing for anti-immunogen antibody, e.g., by ELISA. These and other similar approaches will be apparent to the skilled artisan.

The antigen can be derived and/or isolated from essentially any desired source depending on the infectious disease, autoimmune disease, condition, cancer, pathogen, or a disease that is to be treated with a given vaccine composition. By way of illustration, the antigens can be derived from viral sources, such as influenza virus, feline leukemia virus, feline immunodeficiency virus, Human HIV-1, HIV-2, Herpes Simplex virus type 2, Human cytomegalovirus, Hepatitis A, B, C or E, Respiratory Syncytial virus, human papilloma virus rabies, measles, or hoof and mouth disease viruses. Illustrative antigens can also be derived from bacterial sources, such as anthrax, diphtheria, Lyme disease, malaria, tuberculosis, Leishmaniasis, T. cruzi, Ehrlichia, Candida etc., or from protozoans such as Babeosis bovis or Plasmodium. The antigen(s) will typically be comprised of natural or synthetic amino acids, e.g., in the form of peptides, polypeptides, or proteins, can be comprised of polysaccharides, or can be mixtures thereof. Illustrative antigens can be isolated from natural sources, synthesized by means of solid phase synthesis, or can be obtained by way of recombinant DNA techniques.

In another embodiment, tumor antigens are used in the vaccine compositions of the present invention for the prophylaxis and/or therapy of cancer. Tumor antigens are surface molecules that are differentially expressed in tumor cells relative to non-tumor tissues. Tumor antigens make tumor cells immunologically distinct from normal cells and provide diagnostic and therapeutic targets for human cancers. Tumor antigens have been characterized either as membrane proteins or as altered carbohydrate molecules of glycoproteins or glycolipids on the cell surface. Cancer cells often have distinctive tumor antigens on their surfaces, such as truncated epidermal growth factor, folate binding protein, epithelial mucins, melanoferrin, carcinoembryonic antigen, prostate-specific membrane antigen, HER²-neu, which are candidates for use in therapeutic cancer vaccines. Because tumor antigens are normal or related to normal components of the body, the immune system often fails to mount an effective immune response against those antigens to destroy the tumor cells. To achieve such a response, the adjuvant systems described herein can be utilized. As a result, exogenous proteins can enter the pathway for processing endogenous antigens, leading to the production of cytolytic or cytotoxic T cells (CTL). This adjuvant effect facilitates the production of antigen specific CTLs which seek and destroy those tumor cells carrying on their surface the tumor antigen(s) used for immunization. Illustrative cancer types for which this approach can be used include prostate, colon, breast, ovarian, pancreatic, brain, head and neck, melanoma, leukemia, lymphoma, etc.

In one embodiment, the antigen present in the vaccine composition is not a foreign antigen, but a self-antigen, i.e., the vaccine composition is directed toward an autoimmune disease. Examples of autoimmune diseases include type 1 diabetes, conventional organ specific autoimmunity, neurological disease, rheumatic diseases/connective tissue disease, autoimmune cytopenias, and related autoimmune diseases. Such conventional organ specific autoimmunity may include thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo. Such neurological diseases may include schizophrenia, Alzheimer's disease, depression, hypopituitarism, diabetes insipidus, sicca syndrome and multiple sclerosis. Such rheumatic diseases/connective tissue diseases may include rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjögren's syndrome. Other autoimmune related diseases may include autoimmune uvoretinitis, glomerulonephritis, post myocardial infarction cardiotomy syndrome, pulmonary hemosiderosis, amyloidosis, sarcoidosis, aphthous stomatitis, and other immune related diseases, as presented herein and known in the related arts.

In one embodiment, the antigen is covalently bonded to an adjuvant such as the compound of Formula I to produce a discrete molecule which exhibits a surprisingly unexpected enhanced adjuvanting effect on the antigen which is greater than the adjuvanting effect attainable in the absence of such covalent bonding, as in a mixture of components (i.e., the antigen, an AGP, and a saponin). The covalent bonding can be achieved by reaction through functional groups; for example in the case of the compound of Formula I through a carboxylic acid group, a hydroxyl group or an aldehyde functionality. A further enhanced adjuvanting effect may be attained for such covalently-bonded antigen by incorporating a mineral salt adjuvant with such compounds. The mineral salt adjuvant preferably comprises aluminum hydroxide or aluminum phosphate, although other known mineral salt adjuvants, such as calcium phosphate, zinc hydroxide or calcium hydroxide, may be used.

The adjuvant may include other polynucleotides and/or polypeptides. It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

The vaccine compositions of the present invention may be formulated for any appropriate manner of administration, and thus adminstered, including for example, topical, oral, nasal, intravenous, intravaginal, epicutaneous, sublingual, intracranial, intradermal, intraperitoneal, subcutaneous, intramuscular administration, or via inhalation. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable micro spheres are disclosed, for example, in U.S. Pat. Nos. 4,897,268; 5,075,109; 5,928,647; 5,811,128; 5,820,883; 5,853,763; 5,814,344 and 5,942,252, the disclosures of which are incorporated herein by reference in their entireties. Modified hepatitis B core protein carrier systems are also suitable, such as those described in WO 99/40934, and references cited therein, all incorporated herein by reference. One can also employ a carrier comprising particulate-protein complexes, such as those described in U.S. Pat. No. 5,928,647, the disclosure of which is incorporated herein by reference in its entirety, which are capable of inducing a class I-restricted cytotoxic T lymphocyte responses in a host.

In one illustrative embodiment, the vaccine formulations are administered to the mucosae, in particular to the oral cavity, and preferably to a sublingual site, for eliciting an immune response. Oral cavity administration may be preferred in many instances over traditional parenteral delivery due to the ease and convenience offered by noninvasive administration techniques. Moreover, this approach further provides a means for eliciting mucosal immunity, which can often be difficult to achieve with traditional parenteral delivery, and which can provide protection from airborne pathogens and/or allergens. An additional advantage of oral cavity administration is that patient compliance may be improved with sublingual vaccine delivery, especially for pediatric applications, or for applications traditionally requiring numerous injections over a prolonged period of time, such as with allergy desensitization therapies.

The vaccine compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, vaccine compositions of the present invention may be formulated as a lyophilisate. Compounds may also be encapsulated within liposomes using well known technology.

The vaccine compositions of the present invention may also comprise other adjuvants or immunoeffectors. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO₄)₂, AlNa(SO₄)₂, AlNH₄(SO₄), and Al(OH)₃; salts of calcium (e.g, Ca₃(PO₄)₂), iron or zinc; an insoluble suspension of acylated tyrosine; acylated sugars; cationically or anionically derivatized polysaccharides; polynucleotides (for example, poly IC and poly AU acids); polyphosphazenes; cyanoacrylates; polymerase-(DL-lactide-co-glycoside); biodegradable microspheres; liposomes ; lipid A and its derivatives; monophosphoryl lipid A; wax D from Mycobacterium tuberculosis, as well as substances found in Corynebacterium parvum, Bordetella pertussis, and members of the genus Brucella); bovine serum albumin; diphtheria toxoid; tetanus toxoid; edestin; keyhole-limpet hemocyanin; Pseudomonal Toxin A; choleragenoid; cholera toxin; pertussis toxin; viral proteins; and Quil A. Aminoalkyl glucosamine phosphate compounds can also be used (see, e.g., WO 98/50399, U.S. Pat. No. 6,113,918 (which issued from U.S. Ser. No. 08/853,826), and U.S. Ser. No. 09/074,720). In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule. Other known immunostimulatory macromolecules which can be used in the practice of the invention include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (See, Sela, M., Science 166: 1365-1374 (1969)) or glycolipids, lipids or carbohydrates.

Within the vaccine compositions provided herein, the adjuvant composition is preferably designed to induce an immune response predominantly of the Th1 type. High levels of Th1-type cytokines (e.g., IFN-γ, TNF-α, IL-2 and IL-12) tend to favor the induction of cell mediated immune responses to an administered antigen. In contrast, high levels of Th2-type cytokines (e.g., IL-4, IL-S, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see, Mosmann and Coffman, Ann. Rev. Immunol. 1989, 7: 145-173.

The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation will vary depending upon the site of implantation, the rate and expected duration of release and the nature of the condition to be treated or prevented.

Any of a variety of known delivery vehicles may be employed within pharmaceutical compositions and vaccines to facilitate production of an antigen-specific immune response that targets cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-target effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see, Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see, Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding an antigen polypeptide (or portion or other variant thereof) such that the antigen polypeptide, or an immunogenic portion thereof, is expressed on the cell surface. Such transfection may take place ex vivo, and a composition or vaccine comprising such transfected cells, and the adjuvants described herein, may then be used for therapeutic purposes. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the antigen polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

In one embodiment, the vaccine composition comprises a liposome vesicle comprising the compound of Formula I. Liposomes are generally produced from phospholipids or other lipid substances. Procedures for the preparation of liposomes are well known to those of skill in the art. Any lipid capable of forming vesicles that comprises the compound of Formula I can be employed. For clinical application, it is desirable that the lipid be non-toxic, physiologically acceptable, and metabolizable. Common bilayer forming lipids having clinical potential are phospholipids, fatty acids, sphingolipids, glycosphingolipids, and steroids. Glycerol containing phospholipids are the most commonly used component of liposome formulations having clinical utility. One commonly used example is phosphatidylcholine or lecithin. The steroid cholesterol and its derivatives are often included as components of liposomal membranes. The tendency of liposomes to aggregate and fuse can be controlled by the inclusion of small amounts of acidic or basic lipids in the formulation. The properties of liposomes containing phospholipids are determined by the chemistry of the phospholipid. Important considerations are the hydrocarbon chain length, degree of unsaturation of the hydrocarbon chain, degree of branching of the hydrocarbon chain, and temperature of the system.

Multilamellar liposomes can be created by depositing a mixture of lipids as a thin film by evaporation under reduced pressure followed by dispersion with an excess volume of aqueous buffer containing the antigen with or without organic solvents. Another method is to mix the aqueous phase containing the antigen with small unilamellar liposomes followed by lyophilization. The multilamellar liposomes are formed when the lyophilized product is rehydrated, usually with a small amount of distilled water. The small unilamellar liposomes to be used in this process are produced by dispersing the lipids in an aqueous medium followed by a mechanical means of dispersion such as sonication, use of a high pressure device, or a solvent injection method. Large and intermediate sized unilamellar liposomes can also be produced by conventional techniques including detergent dialysis, extrusion through small pore size membranes under high pressure, freeze thawing followed by slow swelling, dehydration followed by rehydration and dilution, or dialysis of lipids in the presence of chaotropic ions. The size of the liposomes can be made more uniform by fractionation procedures such as centrifugation or size exclusion chromatography, homogenization, or capillary pore membrane extrusion.

These vaccines can be used in methods for inducing or enhancing immunogenicity of an antigen in a mammal comprising administering to the mammal a vaccine composition comprising the antigen and an effective amount of a vaccine adjuvant composition comprising an AGP (e.g., a compound of Formula I) and a saponin (e.g., QS-21, a compound of Formulae II, IIa, or IIb etc). As used in this context, the “vaccine adjuvant composition” includes any composition comprising the compound of Formula I that enhances an immune response to an exogenous antigen. Such “vaccine adjuvant composition” includes biodegradable microspheres (e.g., polylactic galactide) and liposomes. See, e.g., Fullerton, U.S. Pat. No. 4,235,877. Vaccine preparation is generally described in, for example, M. F. Powell and M. J. Newman, eds., “Vaccine Design (the subunit and adjuvant approach),” Plenum Press (NY, 1995). Vaccines may be designed to generate antibody immunity and/or cellular immunity.

### EXAMPLES

The following examples are offered to illustrate, but not to limit the claimed invention.

### Example 1

This experiment demonstrates induction of immune responses against a recombinant polypeptide antigen from M. tuberculosis, referred to as rDPV (rMtb 8.4) (Coler et al. (1998) J. Immunol. 161:2356-2364). C57BL/6 mice were vaccinated by i.d. (intradermal) injection with vaccines containing rDPV antigen in 2% oil-in-water emulsions. The dose for the adjuvants was 10 μg Quil A and 5 μg of MPL, or compound B19 or B3 defined above. Control mice were immunized intradermally with SE (oil emulsion) vehicle or intramuscularly with DPV DNA. Each combination of MPL-SE, B19-SE or B3-SE with Quil A mediated enhanced CTL activity (Table 1) measured in the standard chromium (⁵¹ Cr) release assay (see, e.g., Moore et al., (1988) Cell 55: 777-785). The letters SE refer to an oil emulsion formulation.

CTL activity was also analyzed by flow cytometry and intracellular IFNγ expression was evaluated. Effector cells stimulated with either EL 4-DPV target cells or DPV peptides were compared. Both methods of stimulation resulted in similar results. Single cell suspensions of splenocytes were stimulated in vitro with EL4-DPV target cells or DPV peptides. Thirteen days later these cells were assayed for CTL activity against EL-4-DPV or DPV peptides by standard chromium release techniques. The results are depicted in Table 1.

The data indicated that as many as 1 in 20 to 1 in 150 splenic CD8+ lymphocytes were specific for rDPV antigens following vaccination with Quil A plus MPL, or compound B19- or B3-adjuvanted vaccines. These data correlate with the results from the chromium release assay. The comparisons of the intracellular cytokine staining and chromium release assays are depicted in Table 2. To carry out the IFN-γ assay, fresh splenocytes were stimulated in vitro with 5 μg/ml rDPV and supernatants were harvested 3 days later and assayed for IFN-γ by ELISA. The concentration of IFN-γ was measured in 3-day supernatants by ELISA, and is expressed as mean concentration for groups of four mouse spleens.

TABLE 1
Evaluation of Formulations Containing Quil-A and MPL- or
AGP-SE: CTL Response

| | Percent Specific Lysis^{b} | | | |
|---|---|---|---|---|
| Group^{a} | 50:1 | 25:1 | 12.5:1 | 6.25:1 |
| Assay 13 days post tertiary | | | | |
| Nonimmune | 0 | 0 | 0 | 0 |
| Vehicle-SE | 5 | 3 | 2 | 0 |
| Quil-A + Vehicle-SE | 35 | 25 | 16 | 7 |
| Quil-A + MPL-SE | 55 | 40 | 28 | 16 |
| Quil-A + B19-SE | 53 | 50 | 35 | 21 |
| Quil-A + B3-SE | 55 | 43 | 25 | 15 |
| DNA Control | 67 | 66 | 50 | 35 |

^{a}Female C57BL/6 mice were vaccinated with 10 μg rDPV ± 10 μg Quil-A ± 5 μg MPL or AGP by intradermal injection on days 0, 14, and 21. Effector cells were stimulated for 4 days with irradiated DPV-EL4 cells prior to the chromium release assay.
^{b}The percent specific lysis is a measure of the percent chromium release from DPV-EL-4 target cells minus the percent chromium release from EL-4 control cells (nonDPV expressing).

TABLE 2
Evaluation of Formulations Containing Quil-A and MPL- or AGP-SE:
CTL Analysis by Intracellular Cytokine Staining and Comparison
with Chromium Release Assay.

| | CD8⁺IFNγ^{+b} | | Percent Specific Lysis^{c} | |
|---|---|---|---|---|
| | EL4-DPV | DPV Peptide | EL4-DPV | DPV Peptide |
| Group^{a} | Activated | Activated | Activated | Activated |
| Nonimmune | 1:10084 | 1:3496 | 0 | 0 |
| Vehicle-SE | 1:1192 | 1:1084 | 5 | 4 |
| Quil-A + Vehicle- | 1:431 | 1:188 | 35 | 13 |
| SE | | | | |
| Quil-A + MPL-SE | 1:151 | 1:76 | 55 | 5 |
| Quil-A + B19-SE | 1:40 | 1:23 | 53 | 33 |
| Quil-A + B3-SE | 1:80 | 1:50 | 55 | 50 |
| DNA Control | 1:106 | 1:48 | 67 | 7 |

^{a}Female C57BL/6 mice were vaccinated with 10 μg rDPV ± 10 μg Quil-A ± 5 ug MPL or AGP by intradermal injection on days 0, 14, and 21. Effector cells were stimulated for 4 days with irradiated DPV-EL4 cells prior to the chromium release assay.
^{b}Effector cells were stimulated for 5.5 hours with irradiated DPV-EL4 cells or DPV peptides prior to staining for flow cytometry. The values represent the ratios of CD8⁺IFNγ⁺ cells per total CD8⁺ cells.
^{c}Effector cells were stimulated for 4 days with irradiated DPV-EL4 cells or DPV peptides prior to the chromium release assay. The percent specific lysis for the 50:1 effector:target cell ratios is given.

### Example 2

The adjuvant activity of isotucaresol, the compound of formula (IV) above, with or without B19-AF (aqueous formulation of 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-β-D-glucopyranoside, triethylammonium salt) was analyzed. BALB/c mice were vaccinated on days 0 and 21 by s.c. administration of 1 μg rHBsAg±1 mg isotucaresol±5 μg compound B19. The aqueous formulations comprise DPPC surfactant. The antibody titers of IgG, IgG1, IgG2a, and IgG2b were determined using ELISA. There was an apparent synergistic action on antibody titers when isotucaresol and compound B19 were formulated together. The synergy was found with all antibody isotypes examined except IgG1. As a stand-alone adjuvant at a dose of 1 mg, isotucaresol mediated elevated levels of antibody although substantially less than those induced by B19 (Table 3). The antibody response promoted by isotucaresol was indicative of TH2 cytokine help, as the IgG2a/IgG1 ratio was less than that observed in the antigen/PBS control group. Characteristically, B19 increased the ratio of IgG2a/IgG1 and the combination of the two adjuvants overwhelmingly biased the response towards IgG2a. The CTL activity was determined by chromium release assay against transfected target cells expressing MCH restricted HBsAg epitopes. At E:T ratios from 25:1 to 6.25:1, the combination of isotucaresol and compound B19 exhibited a higher percent specific lysis than either compound alone.

TABLE 3
Evaluation of Isotucaresol and Compound B19: Humoral Response.

| | Adjuvant | | | | |
|---|---|---|---|---|---|
| | Dose | Serum Titers^{b} | | | |
| Group^{a} | (μg) | IgG | IgG1 | IgG2a | IgG2b |
| Nonimmune | — | 57 | 50 | 57 | 50 |
| PBS | — | 25,703 | 44,668 | 5,623 | 794 |
| isotucaresol | 1000 | 134,896 | 269,153 | 19,498 | 6,456 |
| isotucaresol/B19 | 1000/5 | 1,621,810 | 407,380 | 1,230,268 | 177,827 |
| B19 | 5 | 537,031 | 354,813 | 269,153 | 58,884 |

^{a}Female BALB/c mice were vaccinated with 1 μg rHBsAg ± adjuvants by subcutaneous injection on days 0, and 21.
^{b}The geometric mean titers for HBsAg-specific antibodies were determined from serum collected 21 days post the secondary vaccination.

### Example 3

Three isotucaresol derivatives, isotucaresol methyl ester (“compound B5”), O-carboxymethyl isotucaresol (“Compound B6”), and O-carboxypropyl isotucaresol (“Compound B7”) were evaluated in this example. This study looked at doses of 1000, 500 and 250 μg/mouse in order to find optimal doses. These doses were chosen based on tucaresol, a chemically related drug, which has an optimal dose of approximately 1 mg/mouse. The antibody titers and CTL assays were carried out as in Example 2.

Additionally, Compound B19 was combined with 500 ug of the isotucaresol derivatives to determine if any synergy resulted from the mixtures. Similar to isotucaresol itself, the three derivatives all induced humoral responses characteristic of TH-2 cytokine help resulting in greater enhancement of the IgG1 isotype. Overall the lower adjuvant doses (250 μg/mouse) stimulated the strongest antibody responses and of the 3 adjuvants, isotucaresol methyl ester (B5) induced the highest titers of the 3 compounds (Table 4). In each case mixing the isotucaresol derivatives with compound B19 resulted in higher titers than the derivatives induced by themselves, although B19 by itself produced the strongest antibody responses.

TABLE 4
Evaluation of Isotucaresol Methyl Ester(B5, O-Carboxymethyl
Isotucaresol, (B6) and O-carboxypropyl Isotucaresol, (B7):
Humoral Response.

| | Adjuvant | | | | |
|---|---|---|---|---|---|
| | Dose | Serum Titers^{b} | | | |
| Group^{a} | (μg) | IgG | IgG1 | IgG2a | IgG2b |
| Nonimmune | — | 50 | 50 | 50 | 50 |
| PBS | — | 3801 | 12,882 | 239 | 199 |
| B5 | 1000 | 64,565 | 154,881 | 8128 | 1412 |
| B5 | 500 | 32,359 | 109,647 | 2884 | 1412 |
| B5 | 250 | 45,708 | 77,624 | 19,054 | 1995 |
| B5/B19 | 500/5 | 309,029 | 64,565 | 181,970 | 26,915 |
| B6 | 1000 | 8128 | 32,359 | 602 | 602 |
| B6 | 500 | 4,786 | 11,481 | 1412 | 707 |
| B6 | 250 | 16,218 | 77,624 | 1995 | 602 |
| B6/B19 | 500/5 | 154,881 | 218,776 | 64,565 | 13,489 |
| B7 | 1000 | 5754 | 11,481 | 1202 | 851 |
| B7 | 500 | 6760 | 16,218 | 1412 | 851 |
| B7 | 250 | 13,489 | 45,708 | 1412 | 851 |
| B7/B19 | 500/5 | 257,039 | 38,018 | 128,824 | 38,018 |
| B19 | 5 | 363,078 | 64,565 | 309,029 | 45,708 |

^{a}Female BALB/c mice were vaccinated with 1 μg rHBsAg ± adjuvants by subcutaneous injection on days 0, and 21.
^{b}The geometric mean titers for HBsAg-specific antibodies were determined from serum collected 21 days post the secondary vaccination.

The vaccine containing 250 μg ofB5, isotucaresol methyl ester, induced a strong CTL response in this model, 69% specific lysis at the 50:1 E:T ratio. CTL activity was enhanced even more when B19 was blended with it. B6, O-carboxymethyl isotucaresol, mediated only low levels of activity, about 30-35% specific lysis at the 50:1 E:T ratio, independent of the dose administered. When B19 and B6 were combined a stronger CTL response was elicited. A 1 mg dose of the third compound B7, O-carboxypropyl isotucaresol, gave the strongest response in this experiment with 74% specific lysis at the 50:1:T ratio.

### Example 4

This example shows the efficacy of the semisynthetic triterpenoid saponin derivative, GPI-0100, in combination with AGPs of the the invention (MPL, B19 or B3). For these experiments, 6 mice per group were immunized subcutaneously (SC) three times with 21 days between primary, secondary and tertiary immunizations using the mycobacterial antigen DPV at 10 μg per dose. Spleens and peripheral blood were harvested at 2 weeks following the secondary and tertiary immunizations and evaluated by measuring DPV specific serum IgG₁, IgG_{2b} levels, IFNγ release from rDPV activated spleen cell cultures, intracellular cytokine (ICC) for IFNγ and CTL activity using standard assays. Briefly, no significant T-cell immune responses were detected with any of the adjuvant combinations tested following two subcutaneous immunizations. However, following the third immunization, CD8 T-cell immune responses were observed in the GPI-0100+B139 and GPI-0100+B3. Serum IgG₁ and IgG_{2b} DPV-specific antibodies were detected following both the secondary and tertiary immunizations in all groups with the highest responses in groups immunized with GPI-0100+AGPs. Based on these data it was determined that a combination of GPI-0100 and AGPs induces a synergistic immune response.

### Example 5

This example shows that GPI-0100 in combination with B19-AF mediates vaccine antigen-specific immunity. For this experiment, 6 mice per group were immunized SC three times with 3-4 weeks between primary, secondary and tertiary immunizations using 50, 100 or 250 μg of GPI-0100, 10 μg DPV and 10 μg B19. Mice were harvested at 2 weeks following each boost and evaluated by measuring DPV specific serum IgG₁, IgG_{2b} levels, IFNγ release from rDPV activated spleen cell cultures, ICC for IFNγ and CTL activity. No significant T-cell response was detected to any of the rDPV+adjuvant combinations following two or three immunizations. In contrast, serum anti-rDPV IgG₁ and IgG_{2b} antibody levels were enhanced in mice immunized with B19+GPI-0100, as compared to the adjuvants used individually.

### Example 6

This example shows that increasing doses of GPI-0100 both individually and in combination with B19-AF for mediating vaccine antigen-specific immunity. For this experiment, 6 mice per group were immunized SC three times with 3-4 weeks between primary, secondary and tertiary immunizations using 50, 100 or 250 μg of GPI-0100, 10 μg DPV and 10 μg B19. Mice were harvested at 2 weeks following each boost and evaluated by measuring DPV specific serum IgG₁, IgG_{2b} levels, IFNγ release from rDPV activated spleen cell cultures, ICC for IFNγ and CTL activity. No significant T-cell response was detected to any of the rDPV+adjuvant combinations following two or three immunizations (data not shown). In contrast, serum anti-rDPV IgG₁ and IgG_{2b} antibody levels were enhanced in mice immunized with B19+GPI-0100 at all dosage levels.

### Example 7

This example shows the efficacy of GPI-0100 at 25, 50, 100 and 200 μg, in combination with B19-SE (5 μg) for mediating vaccine antigen-specific immunity to a hepatitis vaccine. For this experiment, 10 mice per group were immunized SC three times on days 0, 21, and 57 with 1 μg rHBsAg. After the secondary vaccination significant CTL activity was observed in the GPI-0100 groups. However, the vehicle control group (no adjuvant) also showed substantial activity. Following the third vaccination no substantial CTL activity was evident from any group. The humoral response was also evaluated following the first and the second vaccination. These results indicated GPI-0100 induced very strong antibody responses. GPI-0100 in combination with B19-SE polarize d the antibody response toward production of IgG2a and IgG2b.

### Example 8

This example shows the efficacy of GPI-0100 (100 μg) in combination with MPL-AF, B19-AF, B9-AF or B3-AF for mediating cellular and humoral responses to the mycobacterial antigen MTCC-2. For this experiment, 4 mice per group were immunized SC three times on days 0, 21, and 42 with 5 μg rMTCC-2. Four weeks after the third vaccination spleen cells and serum were harvested for evaluation. Interferon production from splenocytes stimulated in culture with MTCC-2 was enhanced markedly in groups vaccinated with the combination of GPI-0100 and MPL, B19, B9 or B3. The antibody responses were not enhanced over those induced with B19, B9 or B3 alone.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

What is claimed is:

1. An immunostimulant composition comprising:
(a) at least one aminoalkyl glucosaminide phosphate (AGP); and
(b) at least one saponin.

2. The composition of claim 1, wherein the AGP comprises a compound having the structure: 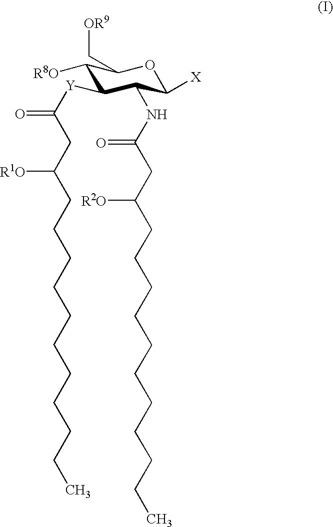
and pharmaceutically acceptable salts and derivatives thereof, wherein Y is —O— or —NH—; R¹ and R² are each independently selected from saturated and unsaturated (C₂-C₂₄) aliphatic acyl groups; R⁸ is —H or —PO₃R¹¹R¹², wherein R¹¹ and R¹² are each independently —H or (C₁-C₄) aliphatic groups; R⁹ is —H, —CH₃ or —PO₃R¹³R¹⁴ wherein R¹³ and R¹⁴ are each independently selected from —H and (C₁-C₄) aliphatic groups; and wherein at least one of R⁸ and R⁹ is a phosphorus-containing group, but R⁸ and R⁹ are not both phosphorus-containing groups; and X is a group selected from the formulae: 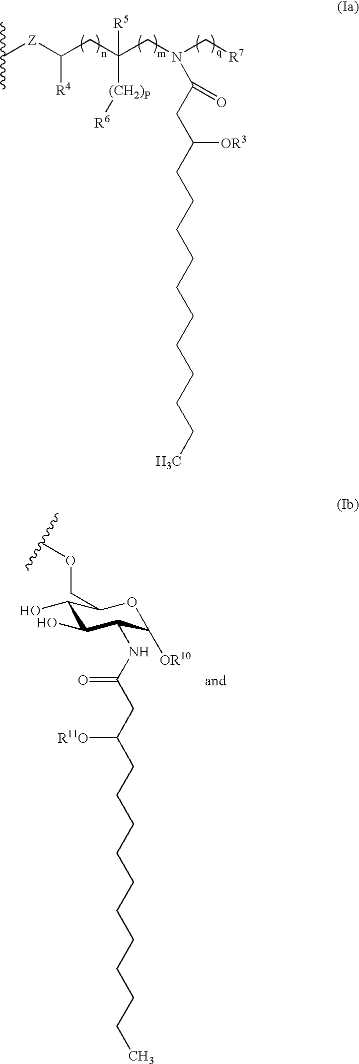 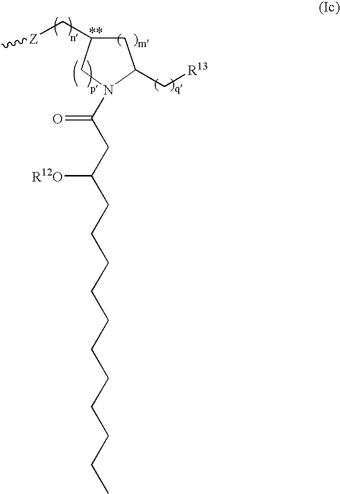
wherein the subscripts n, m, p, q, n′, m′, p′ and q′ are each independently an integer of from 0 to 6, provided that the sum of p′ and m′ is an integer from 0 to 6; R³, R¹¹, and R¹² are independently a saturated or unsaturated optionally substituted aliphatic (C₂-C₂₄)acyl group, provided that when X is formula (Ia), one of R¹, R² and R³is optionally hydrogen; R⁴ and R⁵ are independently selected from H and methyl; R⁶ and R⁷ are independently selected from H, OH, (C₁-C₄)oxyaliphatic groups, —PO₃H₂, —OPO₃H₂, —SO₃H, —OSO₃H, —NR¹⁵R¹⁶, —SR¹⁵, —CN, —NO₂, —CHO, —CO₂R¹⁵, —CONR¹⁵R¹⁶, —PO₃R¹⁵R¹⁶, —OPO₃R¹⁵R¹⁶, —SO₃R¹⁵ and —OSO₃R¹⁵, wherein R¹⁵ and R¹⁶ are each independently select from H and (C₁-C₄)aliphatic groups; R¹⁰ is selected from H, CH₃, —PO₃H₂, ω-phosphonooxy(C₂-C₂₄)alkyl, and ω-carboxy(C₁-C₂₄)alkyl; R¹³ is independently selected from H, OH, (C₁-C₄)oxyaliphatic groups, —PO₃R¹⁷R¹⁸, —OPO₃R¹⁷R¹⁸, —SO₃R¹⁷, —OSO₃R¹⁷, —NR¹⁷R¹⁸, —SR¹⁷, —CN, —NO₂, —CHO, —CO₂R¹⁷, and —CONR¹⁷R¹⁸, wherein R¹⁷ and R¹⁸ are each independently selected from H and (C₁-C₄)aliphatic groups; and Z is —O— or —S—.

3 The composition of claim 2, wherein X is a group of formula (Ia).

4. The composition of claim 2, wherein X is a group of formula (Ib).

5. The composition of claim 2, wherein X is a group of formula (Ic).

6. The composition of claim 2, wherein X is formula (Ia) and one of R¹, R² and R³ is hydrogen.

7. The composition of claim 2, wherein R¹, R², R³, R¹¹ and R¹² are each acyl.

8. The composition of claim 3, wherein R¹, R² and R³ are each C₇-C₁₆ aliphatic acyl groups.

9. The composition of claim 3, wherein R¹, R² and R³ are each C₈-C₁₄ aliphatic acyl groups.

10. The composition of claim 3, wherein R¹, R² and R³ are each C₉-C₁₄ aliphatic acyl groups.

11. The composition of claim 3, wherein R¹, R² and R³ are each C₁₀-C₁₄ aliphatic acyl groups.

12. The composition of claim 3, wherein R¹, R² and R³ are each C₁₀-C₁₄ saturated aliphatic acyl groups.

13. The composition of claim 5, wherein R¹, R² and R¹² are each C₉-C₁₄ aliphatic acyl groups.

14. The composition of claim 5, wherein R¹, R² and R¹² are each C₁₀-C₁₄ aliphatic acyl groups.

15. The composition of claim 5, wherein R¹, R² and R³ are each C₁₀-C₁₄ saturated aliphatic acyl groups.

16. The composition of claim 2, wherein X is oxygen.

17. The composition of claim 2, wherein R⁸ is a phosphorus-containing group and R⁹ is hydrogen.

18. The composition of claim 2, wherein R⁸ or R⁹ is a phosphorus-containing group, and R¹¹ and R¹², or R¹³ and R¹⁴, respectively, are both hydrogen.

18. The composition of claim 3, wherein the total of n+m is 0, 1, or 2.

19. The composition of claim 3, wherein p and q are independemtly 0, 1 or 2.

20. The composition of claim 3, wherein R⁶ is selected from hydrogen, hydroxy and carboxy.

21. The composition of claim 5, wherein n′, m′, p′ and q′ are idependently 0, 1 or 2.

22. The composition of claim 5, wherein n′ is 1, m′ is 2, and p′ and q′ are zero.

23. The composition of claim 22, wherein R¹, R² and R¹² are each C₁₀-C₁₄ saturated aliphatic acyl groups.

24. The composition of claim 23, wherein Y and Z are both oxygen; R¹³ is hydrogen; and R¹, R² and R¹² are each C₁₀ saturated aliphatic acyl groups.

25. The composition of claim 23, wherein Y and Z are both oxygen; R¹³ is hydrogen; and R¹, R² and R¹² are each C₁₂ saturated aliphatic acyl groups.

26. The composition of claim 23, wherein Y and Z are both oxygen; R¹³ is hydrogen; and R¹, R² and R¹² are each C₁₄ saturated aliphatic acyl groups.

27. The composition of claim 1, wherein the AGP is a monophosphoryl lipid A.

28. The composition of claim 3, wherein R¹, R² and R³ all are n-C₁₃H₂₇CO; X and Y are both oxygen; n, m, p, and q are each zero; R⁴, R⁵, R⁶, R⁷ and R⁹ are each hydrogen; and R⁸ is PO₃H₂.

29. The composition of claim 3, wherein R¹, R² and R³ all are n-C₁₁H₂₃CO; X and Y are both oxygen; n, m, and q are each zero; p is 1; R⁴, R⁵, R⁷ and R⁹ are each hydrogen; R⁶ is hydroxy; and R⁸ is PO₃H₂.

30. The composition of claim 1 wherein the saponin is selected from naturally obtained saponins, synthetically obtained saponins, saponin conjugates, saponin derivatives, and saponin mimetics.

31. The composition of claim 1, wherein the saponin comprises a Quillaja saponin.

32. The composition of claim 31, wherein the Quillaja saponin comprises Quil A, QS-7, QS-17, QS-18 or QS-21.

33. The composition of claim 1, wherein the saponin comprises a triterpene saponin-lipophile conjugate comprising a nonacylated or desacylated triterpene saponin that includes a 3-glucuronic acid residue; and a lipophilic moiety; wherein said saponin and said lipophilic moiety are covalently attached to one another, either directly or through a linker group, and wherein said direct attachment or attachment to said linker occurs through a covalent bond between the carboxyl carbon of said 3-glucuronic acid residue and a suitable functional group on the lipophilic residue or linker group.

34. The composition of claim 33, wherein the triterpene saponin (a) has a triterpene aglycone core structure with branched sugar chains attached to positions 3 and 28, and an aldehyde group linked or attached to position 4; and (b) is either originally non-acylated, or requires removal of an acyl or acyloyl group that is bound to a saccharide at the 28-position of the triterpene aglycone

34. The composition of claim 33, wherein said lipophilic moiety comprises one or more residues of a fatty acid, terpenoid, aliphatic amine, aliphatic alcohol, aliphatic mercapton mono- or poly-C₂-C₄ alkyleneoxy derivative of a fatty acid, mono- or poly-C₂-C₄ alkyleneoxy derivative of a fatty alcohol, glycosyl-fatty acid, glycolipid, phospholipid or a mono-, or di-acylglycerol.

35. The composition of claim 1, wherein the saponin comprises GPI-0100.

36. The composition of claim 33, wherein said triterpene saponin has a quillaic acid or gypsogenin core structure.

37. The composition of claim 36, wherein said desacylsaponin or nonacylated saponin is selected from the group consisting of Quillaja desacylsaponin, S. jenisseensis desacylsaponin Gypsophila saponin, Saponaria saponin Acanthophyllum saponin and lucyoside P saponin.

38. The composition of claim 1, wherein the saponin comprises a saponin/antigen covalent conjugate composition.

39. The composition of claim 1, wherein the saponin comprises a compound represented by the formula: 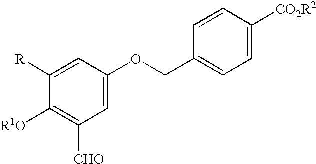
wherein, R is hydrogen or —C(O)H; R¹ is a member selected from the group consisting of hydrogen, an optionally substituted C₁₋₂₀ aliphatic group, a saccharyl group, and a group represented by the formula —C(O)—[C(R³)(R⁴)]ₖ—COOH, wherein each R³ and R⁴ independently is a member selected from the group consisting of hydrogen and optionally substituted C₁₋₁₀ aliphatic groups, and k is a number from 1 to 5; R² is a member selected from the group consisting of hydrogen, an optionally substituted C₁₋₂₀ aliphatic group, and a group represented by the formula —(CH₂)ᵣCH(OH)(CH₂)ₜOR⁵, wherein r and t are independently 1 or 2, and R⁵ is an optionally substituted C₂₋₂₀ aliphatic group, or a group represented by the formula 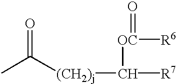
wherein j is 1-5, and R⁶ and R⁷ are independently selected from the group consisting of hydrogen and optionally substituted C₁₋₂₀ aliphatic groups; or a pharmacologically aceptable salt thereof.

40. The composition of claim 39, wherein R¹ is a mono- or disaccharide.

41. The composition of claim 40, wherein R¹ is a glucuronic acid group.

42. The composition of claim 39, wherein R, R¹ and R² are hydrogens.

43. The composition of claim 39, wherein R is hydrogen; R¹ is a saccharyl group, wherein the saccharyl group is a glucuronic acid group; and R² is hydrogen.

44. The composition of claim 39, wherein R is hydrogen; R¹ is represented by the formula —C(O)—[C(R³)(R⁴)]ₖ—COOH wherein R³ and R⁴ are hydrogens and k is 2; and R² is hydrogen.

45. The composition of claim 39, wherein R is hydrogen; R¹ is a saccharyl group, wherein the saccharyl group is a glucuronic acid group; and R² is (CH₂)ᵣCH(OH)(CH₂)ₜOR⁵ wherein r and t are both 1, and R⁵ is an optionally substituted C₂₋₂₀ acyl group.

46. The composition of claim 43, wherein the glucuronic acid group is a β-D-glucuronic acid group.

47. The composition of claim 45, wherein (CH₂)ᵣCH(OH)(CH₂)ₜOR⁵ is a 1-O-acyl-sn-glyceryl group.

48. The composition of claim 47, wherein R⁵ is a member selected from the group consisting of acetyl, octanoyl, and tetradecanoyl groups.

49. The composition of claim 39, wherein R is hydrogen; R¹ is a saccharyl group, wherein the sachharyl group is a glucuronic acid group; and R² is a group represented by the formula: 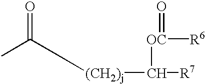
wherein j is 1; R⁶ is an optionally substituted C₁₋₂₀ aliphatic group; and R⁷ is an optionally substituted C₁₋₂₀ aliphatic group.

50. The composition of claim 49, wherein R⁷ is an optionally substituted C₁₁ aliphatic group.

51. The composition of claim 1, further comprising at least one antigen.

52. The composition of claim 51, wherein the antigen is derived from the group consisting of Herpes Simplex Virus type 1, Herpes Simplex virus type 2, Human cytomegalovirus, HIV, Hepatitis A, B, C or E, Respiratory Syncytial virus, human papilloma virus, Influenza virus, Tuberculosis, Leishmaniasis, T.Cruzi, Ehrlichia, Candida, Salmonella, Neisseria, Borrelia, Chlamydia, Bordetella, Plasmodium and Toxoplasma.

53. The composition of claim 51, wherein the antigen is a human tumor antigen.

54. The composition of claim 53, wherein the tumor antigen is derived from a prostate, colon, breast, ovarian, pancreatic, brain, head and neck, melanoma, leukemia or lymphoma cancer.

55. The composition of claim 51, wherein the antigen is a self antigen.

56. The composition of claim 55, wherein the self antigen is an antigen associated with an autoimmune disease.

57. The composition of claim 52, wherein the autoimmune disease is type 1 diabetes, multiple sclerosis, myasthenia gravis, rheumatoid arthritis or psoriasis.

58. The composition of claim 1 comprising an aqueous formulation.

59. The composition of claim 58, wherein the aqueous formulation comprises one or more surfactants.

60. The composition of claim 59, wherein the aqueous formulation comprises one or more phospholipid surfactants.

61. The composition of claim 60, wherein the surfactant is selected from the group consisting of diacyl phosphatidyl glycerols, diacyl phosphatidyl cholines, diacyl phosphatidic acids, and diacyl phosphatidyl ethanolamines.

62. The composition of claim 60, wherein the surfactant is selected from the group consisting of dimyristoyl phosphatidyl glycerol (DPMG), dipalmitoyl phosphatidyl glycerol (DPPG), distearoyl phosphatidyl glycerol (DSPG), dimyristoyl phosphatidylcholine (DPMC), dipalmitoyl phosphatidylcholine (DPPC), distearoyl phosphatidylcholine (DSPC); dimyristoyl phosphatidic acid (DPMA), dipalmitoyl phosphatidic acid (DPPA), distearoyl phosphatidic acid (DSPA); dimyristoyl phosphatidyl ethanolamine (DPME), dipalmitoyl phosphatidyl ethanolamine (DPPE) and distearoyl phosphatidyl ethanolamine (DSPE).

63. The composition of claim 1, comprising an emulsion formulation.

64. The composition of claim 1, comprising a solid formulation.

65. The composition of claim 1, wherein the AGP and saponin are present in synergistically effective amounts.

66. The composition of claim 1, wherein the saponin and AGP are present in a weight ratio of saponin:AGP of from about 1000:1 to about 1:1000.

67. The composition of claim 1 further comprising a vaccine.

68. The composition of claim 2, wherein the saponin is selected from naturally obtained saponins, synthetically obtained saponins, saponin conjugates, saponin derivatives, and saponin mimetics.

69. The composition of claim 3, wherein the saponin is a quillaja saponin.

70. The composition of claim 69, wherein the saponin is QS-21.

71. The composition of claim 3, wherein the saponin is a saponin-lipophile conjugate.

72. The composition of claim 71, wherein the saponin is GPI-0100.

73. The composition of claim 4, wherein the saponin is a quillaja saponin.

74. The composition of claim 73, wherein the saponin is QS-21.

75. The composition of claim 4, wherein the saponin is a saponin-lipophile conjugate.

76. The composition of claim 75, wherein the saponin is GPI-0100.

77. The composition of claim 24, wherein the saponin is QS-21.

78. The composition of claim 24, wherein the saponin is GPI-0100.

79. The composition of claim 25, wherein the saponin is QS-21.

80. The composition of claim 25, wherein the saponin is GPI-0100.

81. The composition of claim 26, wherein the saponin is QS-21.

82. The composition of claim 26, wherein the saponin is GPI-0100.

83. The composition of claim 27, wherein the saponin is a quillaja saponin.

84. The composition of claim 83, wherein the saponin is QS-21.

85. The composition of claim 27, wherein the saponin is a saponin-lipophile conjugate.

86. The composition of claim 85, wherein the saponin is GPI-0100.

87. A method of treating a mammal suffering from or susceptible to a pathogenic infection, cancer or an autoimmune disorder comprising administering to the mammal an effective amount of a composition according to claim 1.

88 A method of treating a mammal suffering from or susceptible to a pathogenic infection, cancer or an autoimmune disorder comprising administering to the mammal an effective amount of a composition according to claim 2

89 A method of treating a mammal suffering from or susceptible to a pathogenic infection, cancer or an autoimmune disorder comprising administering to the mammal an effective amount of a composition according to claim 30.

90. A method of enhancing the immune response in an animal which comprises administering to the animal a composition according to claim 1.

91. A method of enhancing the immune response in an animal which comprises administering to the animal a composition according to claim 2.

92. A method of enhancing the immune response in an animal which comprises administering to the animal a composition according to claim 30.

93. A method of enhancing the immune response in an animal to an antigen which comprises administering to the animal a composition according to claim 1 in combination with an antigen.

94. A method of enhancing the immune response in an animal to an antigen which comprises administering to the animal a composition according to claim 2 in combination with an antigen.

95. A method of enhancing the immune response in an animal to an antigen which comprises administering to the animal a composition according to claim 30 in combination with an antigen.
